(19) United States
(12) Patent Application Publication
(10) Pub. No.: US 2026/0274802 A1
(43) Pub. Date: Sep. 17, 2026

(54) TRPV1 AGONIST AND USES THEREOF

(71) Applicant: SciTherm Therapeutics Corporation, Dover, DE (US)

(72) Inventors: Martín Paul Banda MAGAÑA, Boston, MA (US); Cuauhtémoc Licona CASSANI, Monterrey Nuevo León (MX); Alejandro Torres GAVILÁN, Guadalajara (MX)

(21) Appl. No.: 19/556,713

(22) Filed: Mar. 4, 2026

Related U.S. Application Data
(60) Provisional application No. 63/767,493, filed on Mar. 5, 2025.

(51) Int. Cl. C07D 233/64; A61K 31/165; A61K 31/231; A61K 31/341; A61K 31/36; A61K 31/401; A61K 31/405; A61K 31/4172; A61P 3/00; A61P 3/06; C07C 69/94; C07C 235/34; C07D 207/16; C07D 209/20; C07D 209/42; C07D 307/68; C07D 317/58

(52) U.S. Cl. CPC C07D 233/64; A61K 31/165; A61K 31/231; A61K 31/341; A61K 31/36; A61K 31/401; A61K 31/405; A61K 31/4172; A61P 3/00; A61P 3/06; C07C 69/94; C07C 235/34; C07D 207/16; C07D 209/20; C07D 209/42; C07D 307/68; C07D 317/58

(57) ABSTRACT

The disclosure provides for TRPV1 agonists and methods of use.

### CROSS-REFERENCE TO RELATED APPLICATIONS

This application claims the benefit of U.S. Provisional Application No. 63/767,493 filed Mar. 5, 2025, which is herein incorporated by reference in its entirety.

### FIELD OF THE DISCLOSURE

The disclosure relates to compounds which function as agonists of TRPV1.

### BACKGROUND OF THE DISCLOSURE

The transient receptor potential cation channel subfamily V member 1 (TRPV1) is a protein that is a member of a group of cation channels that are sensitive to vanilloid and capsaicin. TRPV1 provides a sensation of pain (nociception) in response to a variety of exogenous and endogenous physical and chemical stimuli. Its primary function is detection and regulation of body temperature; however, it has been implicated in many physiological processes such as immune response, pain, and organ function.

TRPV1 plays an important role not only in neurons but also in immune cells. Activation of TRPV1 modulates immune response including the release of inflammatory cytokines and chemokines. The expression of TRPV1 has been confirmed in the cells of both innate immunity as well as the cells of adaptive immunity.

Additionally, TRPV1 has implications in cancer therapy. TRPV1 activation caused by its agonist capsaicin has been shown to induce G0-G1 cell arrest and apoptosis in adult T-cell leukemia and multiple myeloma. Capsaicin reduces the expression of anti-apoptotic protein Bcl-2 and it promotes activation of p53, a tumor-suppressor protein known as a major regulator of cell death leading to apoptosis. TRPV1 was found to be overexpressed in several types of cancers including pancreatic cancer and colon adenocarcinoma. This suggests that certain types of cancers might be prone to cell death mediated by vanilloid-induced cell death.

Based on the foregoing, there is a need to identify potent agonists of TRPV1 for the treatment of conditions such as cancer.

### SUMMARY OF THE DISCLOSURE

In an aspect, provided herein is a compound having the structure of Formula (I), or a pharmaceutically acceptable salt or solvate thereof:

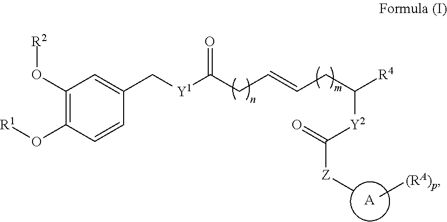

wherein;
Y¹ and Y² are each independently —O— or —NR³—;
Z is absent, optionally substituted C₁-C₂ alkylene, —C═C—, —O—, or —NH—, wherein the alkylene is optionally substituted with —NH₂;
Ring A is an optionally substituted C₆-C₁₀ aryl, optionally substituted 5 to 10-membered heteroaryl, optionally substituted C₃-C₁₀ cycloalkyl, or optionally substituted C₃-C₁₀ heterocycloalkyl, each of which when substituted is substituted with 1 to 6 substituents selected from R^{A};
each R^{A} is independently selected from —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d};
R¹ is hydrogen or C₁-C₆ alkyl which is optionally substituted with 1 to 4 substituents selected from R^{e};
R² is hydrogen, optionally substituted C₁-C₆ alkyl, optionally substituted C₁-C₆ haloalkyl, optionally substituted C₁-C₆ heteroalkyl, or optionally substituted C₁-C₆ hydroxyalkyl, wherein the alkyl, haloalkyl, heteroalkyl, and hydroxyalkyl is optionally substituted with 1 to 4 substituents selected from R^{e};
each R^{e} is independently halogen, —NR^{a}R^{b}, —OR^{c}, or —SR^{a};
or R¹ and R² together with the atoms to which they are attached form a C₃-C₅ heterocycloalkyl;
each R³ is independently hydrogen or —CH₃;
R⁴ is C₄-C₂₀ alkyl;
each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆haloalkyl, optionally substituted C₃-C₁₀ cycloalkyl, and optionally substituted C₃-C₁₀ heterocycloalkyl, each of which is substituted with one or more R^{d};
or R^{a} and R^{b} together with the atoms to which they are attached form a C₃-C₁₀ heterocycloalkyl which is optionally substituted with one or more R^{d};
each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-C₁₀ heterocycloalkyl, optionally substituted C₆-C₁₀ aryl, and optionally substituted 5- to 10-membered heteroaryl, each of which is substituted with one or more R^{d};
or two R^{c} together with the atoms to which they are attached form a C₃-C₁₀ heterocycloalkyl which is optionally substituted with one or more R^{d};
each R^{d} is independently selected from halogen, —OH, —CH₃, —CF₃, —OCH₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m is 1, 2, or 3;
n is an integer from 5-8; and
p is 1-6.

In some embodiments, the compound of Formula (I) has the structure of Formula (II), or a pharmaceutically acceptable salt or solvate thereof:

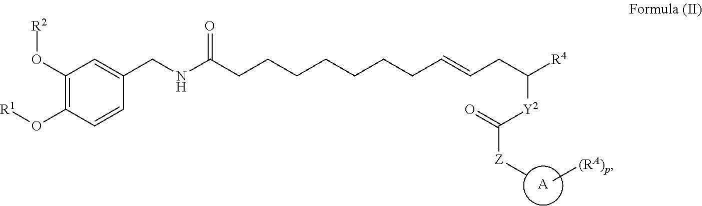

wherein;
Y² is —O— or —NR³—;
Z is absent, optionally substituted C₁-C₂ alkylene, —C═C—, —O—, or —NH—, wherein the alkylene is optionally substituted with —NH₂;
Ring A is an optionally substituted C₆-C₁₀ aryl, optionally substituted 5 to 10-membered heteroaryl, optionally substituted C₃-C₁₀ cycloalkyl, or optionally substituted C₃-C₁₀ heterocycloalkyl, each of which when substituted is substituted with 1 to 6 substituents selected from R^{A}; wherein when Ring A is phenyl then the phenyl is substituted;
each R^{A} is independently selected from —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d};
R¹ is hydrogen or C₁-C₆ alkyl which is optionally substituted with 1 to 4 substituents selected from R^{e};
R² is hydrogen, optionally substituted C₁-C₆ alkyl, optionally substituted C₁-C₆ haloalkyl, optionally substituted C₁-C₆ heteroalkyl, or optionally substituted C₁-C₆ hydroxyalkyl, wherein the alkyl, haloalkyl, heteroalkyl, and hydroxyalkyl is optionally substituted with 1 to 4 substituents selected from R^{e};
or R¹ and R² together with the atoms to which they are attached form a C₃-C₅ heterocycloalkyl;
each R^{e} is independently halogen, —NR^{a}R^{b}, —OR^{c}, or —SR^{a};
R³ is hydrogen or —CH₃;
R⁴ is C₄-C₂₀ alkyl;
each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆haloalkyl, optionally substituted C₃-C₁₀ cycloalkyl, and optionally substituted C₃-C₁₀ heterocycloalkyl, each of which is substituted with one or more R^{d};
or R^{a} and R^{b} together with the atoms to which they are attached form a C₃-C₁₀ heterocycloalkyl which is optionally substituted with one or more R^{d};
each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-Cia heterocycloalkyl, optionally substituted C₆-C₁₀ aryl, and optionally substituted 5- to 10-membered heteroaryl, each of which is substituted with one or more R^{d};
or two R^{c} together with the atoms to which they are attached form a C₃-C₁₀ heterocycloalkyl which is optionally substituted with one or more R^{d};
each R^{d} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃; and
p is 1-6.

In some embodiments, the compound of Formula (II) has the structure of Formula (III), or a pharmaceutically acceptable salt or solvate thereof:

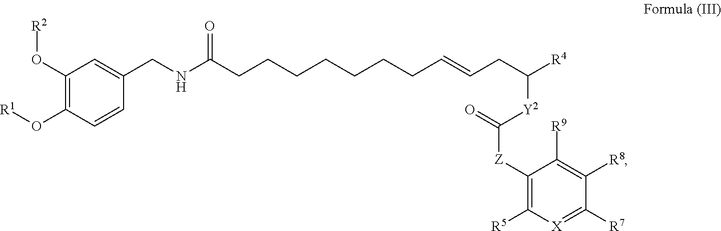

wherein;
X is N or CR⁶;
Y² is —O— or —NR³—;
Z is absent, optionally substituted C₁-C₂ alkylene, —C═C—, —O—, or —NH—, wherein the alkylene is optionally substituted with —NH₂;
R¹ is hydrogen or C₁-C₆ alkyl which is optionally substituted with 1 to 4 substituents selected from R^{e};

R² is hydrogen, C₁-C₆ alkyl, C₁-C₆ haloalkyl, C₁-C₆ heteroalkyl, or C₁-C₆ hydroxyalkyl, wherein the alkyl, haloalkyl, heteroalkyl, and hydroxyalkyl is optionally substituted with 1 to 4 substituents selected from R^{e};
each R^{e} is independently halogen, —NR^{a}R^{b}, —OR^{c}, or —SR^{a};
or R¹ and R² together with the atoms to which they are attached form a C₃-C₅ heterocycloalkyl;
R³ is hydrogen or —CH₃;
R⁴ is C₄-C₂₀ alkyl;
R⁵, R⁶, R⁷, R⁸, and R⁹ are each independently hydrogen, CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d}, wherein at least one of R⁵, R⁶, R⁷, R⁸, or R⁹ is not hydrogen;
or R⁵ and R⁶ or R⁶ and R⁷ or R⁷ and R⁸ together with the atoms to which they are attached form an optionally substituted C₃-C₆ cycloalkyl, optionally substituted C₃-C₆ heterocycloalkyl, optionally substituted C₆-C₈ aryl, or optionally substituted 5 to 6-membered heteroaryl, each of which is optionally substituted with 1 to 4 substituents selected from R^{d};
each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ haloalkyl, optionally substituted C₃-C₁₀ cycloalkyl, and optionally substituted C₃-C₁₀ heterocycloalkyl, each of which is substituted with one or more R^{d};
or R^{a} and R^{b} together with the atoms to which they are attached form a C₃-C₁₀ heterocycloalkyl which is optionally substituted with one or more R^{d};
each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-C₁₀ heterocycloalkyl, optionally substituted C₆-C₁₀ aryl, and optionally substituted 5- to 10-membered heteroaryl, each of which is substituted with one or more R^{d};
or two R^{c} together with the atoms to which they are attached form a C₃-C₁₀ heterocycloalkyl which is optionally substituted with one or more R^{d}; and
each R^{d} is independently selected from halogen, —OH, —CH₃, —CF₃, —OCH₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

In another aspect, provided herein is a pharmaceutical composition comprising a compound of Formula (I), (II), or (III), or a pharmaceutically acceptable salt or solvate thereof; and a pharmaceutically acceptable excipient.

In another aspect, provided herein is a method of treating a disease or condition mediated by transient receptor potential vanilloid 1 (TRPV1) activity in a subject in need thereof, comprising administering to the subject a compound disclosed herein or a pharmaceutically acceptable salt or solvate thereof.

In another aspect, provided herein is a method of treating a metabolic condition in a subject in need thereof, the method comprising administering to the subject a therapeutically effective amount of a compound disclosed herein, or a pharmaceutically acceptable salt or solvate thereof. In some embodiments, the metabolic condition is obesity.

In another aspect, provided herein is a method of treating pain in a subject in need thereof, the method comprising administering to the subject a therapeutically effective amount of a compound disclosed herein, or a pharmaceutically acceptable salt or solvate thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF FIGURES

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1 shows nano-differential scanning calorimetry (Nano-DSC) thermograms of purified TRPV1 in the presence of capsaicin and representative compounds 12, 17, 21, and 24. Heat capacity (Cp) is shown as a function of temperature for TRPV1 incubated with capsaicin or selected compounds at a 1:10 protein-to-ligand molar ratio.

FIG. 2 shows the oxygen consumption rate (OCR) kinetics over 20 h once treatment has started (absolute values). Line chart of raw OCR vs time (0-20 h).

FIG. 3 shows the heatmap of 3D human adipocytes and cardiospheres organoids cell viability.

FIG. 4 shows electrophysiological activity of at TRPV1. The Y-axis displays the normalized response defined as I_max(10 mM compound)/I_(10 mM capsaicin Cap); for convenience, the capsaicin value is set at 1.0. The X-axis lists the evaluated compounds together with capsaicin (positive control).

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Unless defined otherwise, all technical and scientific terms used herein have the same meaning as is commonly understood by one of skill in the art to which this invention belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀alkyl. In some embodiments, the alkyl is a C₁₋₆alkyl. In some embodiments, the alkyl is a C₁-C₅alkyl. In some embodiments, the alkyl is a C₁-C₄alkyl. In some embodiments, the alkyl is a C₁-C₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —C(═O)OH, —C(═O)OMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)=CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —C(═O)OH, C(═O)OMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —C(═O)OH, C(═O)OMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where R^{a} is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —C(═O)OH, C(═O)OMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —C(═O)OH, C(═O)OMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Carbocycle” refers to a saturated, unsaturated, or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated, and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle may be optionally substituted.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —C(═O)OH, C(═O)OMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro, 2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₆ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). In some embodiments, the heterocycloalkyl is a 3- to 8-membered fully saturated heterocycloalkyl. In some embodiments, the heterocycloalkyl is a 3- to 7-membered fully saturated heterocycloalkyl. In some embodiments, the heterocycloalkyl is a 3- to 6-membered fully saturated heterocycloalkyl. In some embodiments, the heterocycloalkyl is a 4- to 6-membered fully saturated heterocycloalkyl. In some embodiments, the heterocycloalkyl is a 5- to 6-membered fully saturated heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —C(═O)OH, C(═O)OMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —C(═O)OH, C(═O)OMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched, and unbranched, carbocyclic, and heterocyclic, aromatic, and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Compounds of the Disclosure

In an aspect, provided herein is a compound having the structure of Formula (I), or a pharmaceutically acceptable salt or solvate thereof:

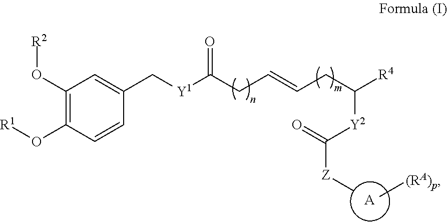

wherein;
Y¹ and Y² are each independently —O— or —NR³—;
Z is absent, optionally substituted C₁-C₂ alkylene, —C═C—, —O—, or —NH—, wherein the alkylene is optionally substituted with —NH₂;
Ring A is an optionally substituted C₆-C₁₀ aryl, optionally substituted 5 to 10-membered heteroaryl, optionally substituted C₃-C₁₀ cycloalkyl, or optionally substituted C₃-C₁₀ heterocycloalkyl, each of which when substituted is substituted with 1 to 6 substituents selected from R^{A};
each R^{A} is independently selected from —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d};
R¹ is hydrogen or C₁-C₆ alkyl which is optionally substituted with 1 to 4 substituents selected from R^{e};
R² is hydrogen, optionally substituted C₁-C₆ alkyl, optionally substituted C₁-C₆ haloalkyl, optionally substituted C₁-C₆ heteroalkyl, or optionally substituted C₁-C₆ hydroxyalkyl, wherein the alkyl, haloalkyl, heteroalkyl, and hydroxyalkyl is optionally substituted with 1 to 4 substituents selected from R^{e};
each R^{e} is independently halogen, —NR^{a}R^{b}, —OR^{c}, or —SR^{a};
or R¹ and R² together with the atoms to which they are attached form a C₃-C₅ heterocycloalkyl;
each R³ is independently hydrogen or —CH₃;
R⁴ is C₄-C₂₀ alkyl;
each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆haloalkyl, optionally substituted C₃-C₁₀ cycloalkyl, and optionally substituted C₃-C₁₀ heterocycloalkyl, each of which is substituted with one or more R^{d};
or R^{a} and R^{b} together with the atoms to which they are attached form a C₃-C₁₀ heterocycloalkyl which is optionally substituted with one or more R^{d};
each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-Cia heterocycloalkyl, optionally substituted C₆-C₁₀ aryl, and optionally substituted 5- to 10-membered heteroaryl, each of which is substituted with one or more R^{d};
or two R^{c} together with the atoms to which they are attached form a C₃-C₁₀ heterocycloalkyl which is optionally substituted with one or more R^{d};
each R^{d} is independently selected from halogen, —OH, —CH₃, —CF₃, —OCH₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m is 1, 2, or 3;
n is an integer from 5-8; and
p is 1-6.

In some embodiments of Formula (I), m is 1. In some embodiments of Formula (I), m is 2. In some embodiments of Formula (I), m is 3.

In some embodiments of Formula (I), n is 6, 7, or 8. In some embodiments of Formula (I), n is 6. In some embodiments of Formula (I), n is 7. In some embodiments of Formula (I), n is 8.

In some embodiments of Formula (I), Y¹ is —O—. In some embodiments of Formula (I), Y¹ is —NR³—. In some embodiments of Formula (I), Y¹ is —NH—.

In some embodiments of Formula (I), when Ring A is phenyl then the phenyl is substituted with 1 to 5 substituents selected from R^{A}.

In some embodiments, provided herein is a compound having the structure of Formula (II), or a pharmaceutically acceptable salt or solvate thereof

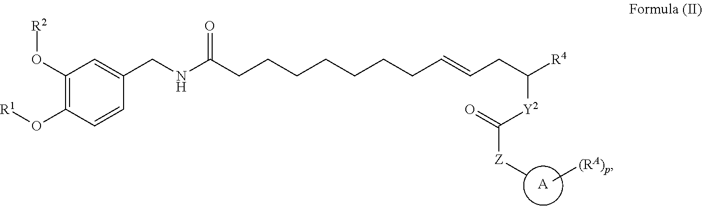

wherein;
Y² is —O— or —NR³—;
Z is absent, optionally substituted C₁-C₂ alkylene, —C═C—, —O—, or —NH—, wherein the alkylene is optionally substituted with —NH₂;
Ring A is an optionally substituted C₆-C₁₀ aryl, optionally substituted 5 to 10-membered heteroaryl, optionally substituted C₃-C₁₀ cycloalkyl, or optionally substituted C₃-C₁₀ heterocycloalkyl, each of which when substituted is substituted with 1 to 6 substituents selected from R^{A}; wherein when Ring A is phenyl then the phenyl is substituted;
each R^{A} is independently selected from —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d};
R¹ is hydrogen or which is C₁-C₆ alkyl optionally substituted with 1 to 4 substituents selected from R^{e};
R² is hydrogen, optionally substituted C₁-C₆ alkyl, optionally substituted C₁-C₆ haloalkyl, optionally substituted C₁-C₆ heteroalkyl, or optionally substituted C₁-C₆ hydroxyalkyl, wherein the alkyl, haloalkyl, heteroalkyl, and hydroxyalkyl is optionally substituted with 1 to 4 substituents selected from R^{e};
or R¹ and R² together with the atoms to which they are attached form a C₃-C₅ heterocycloalkyl;
each R^{e} is independently halogen, —NR^{a}R^{b}, —OR^{c}, or —SR^{a};
R³ is hydrogen or —CH₃;
R⁴ is C₄-C₂₀ alkyl;
each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆haloalkyl, optionally substituted C₃-C₁₀ cycloalkyl, and optionally substituted C₃-C₁₀ heterocycloalkyl, each of which is substituted with one or more R^{d};
or R^{a} and R^{b} together with the atoms to which they are attached form a C₃-C₁₀ heterocycloalkyl which is optionally substituted with one or more R^{d};
each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-C₁₀ heterocycloalkyl, optionally substituted C₆-C₁₀ aryl, and optionally substituted 5- to 10-membered heteroaryl, each of which is substituted with one or more R^{d};
or two R^{c} together with the atoms to which they are attached form a C₃-C₁₀ heterocycloalkyl which is optionally substituted with one or more R^{d};
each R^{d} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃; and
p is 1-6.

In some embodiments of Formula (I) or (II), Ring A is an optionally substituted C₃-C₁₀ cycloalkyl or optionally substituted C₃-C₁₀ heterocycloalkyl, each of which when substituted is substituted with 1 to 6 substituents selected from R^{A}. In some embodiments of Formula (I) or (II), Ring A is an optionally substituted C₃-C₈ cycloalkyl or optionally substituted C₃-C₈ heterocycloalkyl, each of which when substituted is substituted with 1 to 6 substituents selected from R^{A}. In some embodiments of Formula (I) or (II), Ring A is an optionally substituted C₃-C₈ cycloalkyl. In some embodiments of Formula (I) or (II), Ring A is an optionally substituted C₃-C₁₀ heterocycloalkyl comprising 1, 2, or 3 heteroatoms selected from N, O, and S. In some embodiments of Formula (I) or (II), Ring A is an optionally substituted C₃-C₈ heterocycloalkyl comprising 1 or 2 heteroatoms selected from N and O. In some embodiment of Formula (I) or (II), Ring A is pyrrolidine or piperidine.

In some embodiments of Formula (I) or (II), Ring A is an optionally substituted 5 to 10-membered heteroaryl comprising 1, 2, or 3 heteroatoms selected from N, O, and S, which is optionally substituted with 1 to 6 substituents selected from R^{A}. In some embodiments of Formula (I) or (II), Ring A is an optionally substituted 5 to 10-membered heteroaryl comprising 1 or 2 heteroatoms selected from N and 0. In some embodiments of Formula (I) or (II), Ring A is an optionally substituted 5-membered heteroaryl. In some embodiments of Formula (I) or (II), Ring A is furan, pyrazole, pyrrole, triazine, oxazole, thiophene, diazole, indole, or indoline. In some embodiments of Formula (I) or (II), Ring A is furan, pyrazole, or pyrrole. In some embodiments of Formula (I) or (II), Ring A is indole or indoline.

In some embodiments of Formula (I) or (II), Ring A is a C₆-C₁₀ aryl which is substituted with 1 to 6 substituents selected from R^{A}. In some embodiments of Formula (I) or (II), Ring A is phenyl or naphthyl. In some embodiments of Formula (I) or (II), Ring A is phenyl which is substituted with 1 to 3 substituents selected from R^{A}. In some embodiments of Formula (I) or (II), Ring A is naphthalene which is substituted with 1 to 3 substituents selected from R^{A}.

In some embodiments of Formula (I) or (II), Ring A is phenyl, naphthyl, indole, furan, pyrazole, or imidazole. In some embodiments of Formula (I) or (II), Ring A is phenyl or naphthyl. In some embodiments of Formula (I) or (II), Ring A is phenyl. In some embodiments of Formula (I) or (II), Ring A is naphthyl.

In some embodiments of Formula (I) or (II), Ring A is a 5-membered heteroaryl; and Z is C₁-C₂ alkylene, optionally substituted with —NH₂. In some embodiments of Formula (I) or (II), Ring A is pyrrole, indole, or indoline; and Z is —CH₂— or —CH(NH₂)CH₂—.

In some embodiments of Formula (I) or (II), Ring A is substituted phenyl or 5-membered heteroaryl; and Z is absent or —C═C—. In some embodiments, Ring A is substituted phenyl; and Z is absent or —C═C—. In some embodiments of Formula (I) or (II), Ring A is a 5-membered heteroaryl; and Z is absent or —C═C—.

In some embodiments of Formula (I) or (II), each R^{A} is independently selected from —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, or heteroaryl is optionally substituted with 1 to 4 substituents selected from R^{d}. In some embodiments of Formula (I) or (II), each R^{A} is independently selected from —NR^{a}R^{b}, —OR^{c}, or C₆-C₁₀ aryl. In some embodiments, each R^{A} is independently —NR^{a}R^{b}, or —OR^{c}. In some embodiments of Formula (I) or (II), each R^{A} is independently —OH, —OCH₃, or phenyl. In some embodiments of Formula (I) or (II), each R^{A} is independently —OR^{c}, wherein each R^{c} is independently hydrogen or C₁-C₆ alkyl. In some embodiments of Formula (I) or (II), each R^{A} is independently —OH or —OCH₃.

In some embodiments of Formula (II), Y² is —O—; Z is absent, optionally substituted C₁-C₂ alkylene, or —C═C—, wherein the alkylene is optionally substituted with —NH₂; R¹ is hydrogen or C₁-C₆ alkyl; R² is hydrogen, C₁-C₆ alkyl, or C₁-C₆ haloalkyl; R³ is hydrogen; and R⁴ is n-hexyl; Ring A is an optionally substituted C₆-C₁₀ aryl, optionally substituted 5 to 10-membered heteroaryl, optionally substituted C₃-C₁₀ cycloalkyl, or optionally substituted C₃-C₁₀ heterocycloalkyl, each of which when substituted is substituted with 1 to 6 substituents selected from R^{A}; wherein when Ring A is phenyl then the phenyl is substituted; and each R^{A} is independently selected from —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from Rd.

In some embodiments of Formula (II), Y² is —O—; Z is absent, optionally substituted C₁-C₂ alkylene, or —C═C—, wherein the alkylene is optionally substituted with —NH₂; R¹ is hydrogen and R² is —CH₃; R³ is hydrogen; and R⁴ is n-hexyl; Ring A is an optionally substituted C₆-C₁₀ aryl, optionally substituted 5 to 10-membered heteroaryl, optionally substituted C₃-C₁₀ cycloalkyl, or optionally substituted C₃-C₁₀ heterocycloalkyl, each of which when substituted is substituted with 1 to 6 substituents selected from R^{A}; wherein when Ring A is phenyl then the phenyl is substituted; and each R^{A} is independently selected from —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d}.

In some embodiments of Formula (II), Y² is —O—; Z is absent, optionally substituted C₁-C₂ alkylene, or —C═C—, wherein the alkylene is optionally substituted with —NH₂; R¹ is hydrogen and R² is —CH₃; R³ is hydrogen; and R⁴ is n-hexyl; Ring A is an optionally substituted C₆-C₁₀ aryl or optionally substituted 5 to 10-membered heteroaryl, wherein the heteroaryl comprises 1 or 2 heteroatoms selected from N and 0, wherein the aryl and heteroaryl when substituted is substituted with 1 to 6 substituents selected from R^{A}; wherein when Ring A is phenyl then the phenyl is substituted; and each R^{A} is independently selected from —OH or —OCH₃.

In some embodiments, provided herein is a compound having the structure of Formula (III), or a pharmaceutically acceptable salt or solvate thereof:

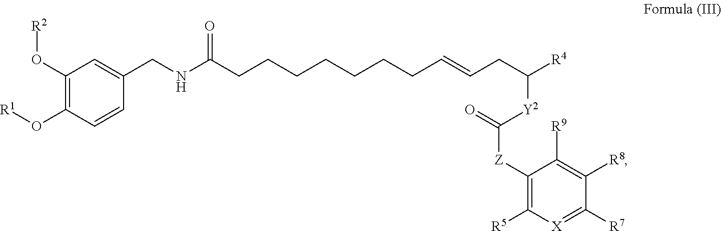

wherein;
X is N or CR⁶;
Y² is —O— or —NR³—;
Z is absent, optionally substituted C₁-C₂ alkylene, —C═C—, —O—, or —NH—, wherein the alkylene is optionally substituted with —NH₂;
R¹ is hydrogen or C₁-C₆ alkyl which is optionally substituted with 1 to 4 substituents selected from R^{e};
R² is hydrogen, optionally substituted C₁-C₆ alkyl, optionally substituted C₁-C₆ haloalkyl, optionally substituted C₁-C₆ heteroalkyl, or optionally substituted C₁-C₆ hydroxyalkyl, wherein the alkyl, haloalkyl, heteroalkyl, and hydroxyalkyl is optionally substituted with 1 to 4 substituents selected from R^{e};
each R^{e} is independently halogen, —NR^{a}R^{b}, —OR^{c}, or —SR^{a};
or R¹ and R² together with the atoms to which they are attached form a C₃-C₅ heterocycloalkyl;
R³ is hydrogen or —CH₃;
R⁴ is C₄-C₂₀ alkyl;
R⁵, R⁶, R⁷, R⁸, and R⁹ are each independently hydrogen, CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d}, wherein at least one of R⁵, R⁶, R⁷, R⁸, or R⁹ is not hydrogen;
or R⁵ and R⁶ or R⁶ and R⁷ or R⁷ and R⁸ together with the atoms to which they are attached form an optionally substituted C₃-C₆ cycloalkyl, optionally substituted C₃-C₆ heteroalkyl, optionally substituted C₆-C₈ aryl, or optionally substituted 5 to 6-membered heteroaryl, each of which is optionally substituted with 1 to 4 substituents selected from R^{d};
each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆haloalkyl, optionally substituted C₃-C₁₀ cycloalkyl, and optionally substituted C₃-C₁₀ heterocycloalkyl, each of which is substituted with one or more R^{d};
or R^{a} and R^{b} together with the atoms to which they are attached form a C₃-C₁₀ heterocycloalkyl which is optionally substituted with one or more R^{d};
each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-C₁₀ heterocycloalkyl, optionally substituted C₆-C₁₀ aryl, and optionally substituted 5- to 10-membered heteroaryl, each of which is substituted with one or more R^{d};
or two R^{c} together with the atoms to which they are attached form a C₃-C₁₀ heterocycloalkyl which is optionally substituted with one or more R^{d}; and
each R^{d} is independently selected from halogen, —OH, —CH₃, —CF₃, —OCH₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

In some embodiments of Formula (III), X is CR⁶; Y² is —O—; Z is absent, optionally substituted C₁-C₂ alkylene, or —C═C—, wherein the alkylene is optionally substituted with —NH₂; R¹ is hydrogen or C₁-C₆ alkyl; R² is hydrogen, C₁-C₆ alkyl, or C₁-C₆ haloalkyl; R³ is hydrogen; and R⁴ is n-hexyl. In some embodiments of Formula (III), X is CR⁶; Y² is —O—; Z is absent, optionally substituted C₁-C₂ alkylene, or —C═C—, wherein the alkylene is optionally substituted with —NH₂; R¹ is hydrogen and R² is —CH₃; R³ is hydrogen; and R⁴ is n-hexyl. In some embodiments of Formula (III), X is CR⁶; Y² is —O—; Z is absent or —C═C—; R¹ is hydrogen and R² is —CH₃; R³ is hydrogen; and R⁴ is n-hexyl.

In some embodiments of Formula (III), X is CR⁶. In some embodiments of Formula (III), X is N.

In some embodiments of Formula (III), R⁵, R⁶, R⁷, R⁸, and R⁹ are each independently hydrogen, CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, or C₁-C₆ hydroxyalkyl, wherein each alkyl, haloalkyl, heteroalkyl, and hydroxyalkyl is optionally substituted with 1 to 4 substituents selected from R^{d}. In some embodiments of Formula (III), R⁵, R⁶, R⁷, R⁸, and R⁹ are each independently hydrogen, —NR^{a}R^{b}, or —OR^{c}. In some embodiments of Formula (III), R⁵, R⁶, R⁷, R⁸, and R⁹ are each independently hydrogen or —OR^{c}. In some embodiments of Formula (III), R⁵, R⁶, R⁷, R⁸, and R⁹ are each independently hydrogen, —OH, or —OCH₃.

In some embodiments of Formula (III), at least one of R⁵, R⁶, R⁷, R⁸, or R⁹ is not hydrogen. In some embodiments of Formula (III), at least two of R⁵, R⁶, R⁷, R⁸, or R⁹ are not hydrogen.

In some embodiments of Formula (III), R⁵, R⁸, and R⁹ are each hydrogen.

In some embodiments of Formula (III), R⁵, R⁶, and R⁹ are each hydrogen.

In some embodiments of Formula (III), R⁶ and R⁷ are each independently —OR^{c}, wherein each R^{c} is independently selected from hydrogen, optionally substituted C₁-C₆ alkyl, or optionally substituted C₁-C₆ haloalkyl. In some embodiments of Formula (III), R⁶ and R⁷ are each independently —OR^{c}, wherein each R^{c} is independently selected from hydrogen or C₁-C₆ alkyl. In some embodiments of Formula (III), R⁶ and R⁷ are each independently —OH or —OCH₃; and R⁵, R⁸, and R⁹ are each hydrogen. In some embodiments of Formula (III), R⁶ is —OH; R⁷ —OCH₃; and R⁵, R⁸, and R⁹ are each hydrogen. In some embodiments of Formula (III), R⁶ is —OCH₃; R⁷ is —OH; and R⁵, R⁸, and R⁹ are each hydrogen.

In some embodiments of Formula (III), R⁷ and R⁸ are each independently —OR^{c}, wherein each R^{c} is independently selected from hydrogen, optionally substituted C₁-C₆ alkyl, or optionally substituted C₁-C₆ haloalkyl. In some embodiments of Formula (III), R⁷ and R⁸ are each independently —OH or —OCH₃; and R⁵, R⁶, and R⁹ are each hydrogen. In some embodiments of Formula (III), R⁷ is —OH; R⁸ —OCH₃; and R⁵, R⁶, and R⁹ are each hydrogen. In some embodiments of Formula (III), R⁷ is —OCH₃; R⁸ is —OH; and R⁵, R⁶, and R⁹ are each hydrogen.

In some embodiments of Formula (III), R⁹ is hydrogen; and R⁵, R⁶, R⁷, and R⁸ are each independently hydrogen or —OR^{c}, wherein each R^{c} is independently C₁-C₆ alkyl, wherein at least one of R⁵, R⁶, R⁷, or R⁸ is not hydrogen. In some embodiments of Formula (III), R⁹ is hydrogen; and R⁵, R⁶, R⁷, and R⁸ are each independently hydrogen, —OH, or —OCH₃, wherein at least one of R⁵, R⁶, R⁷, or R⁸ is not hydrogen. In some embodiments of Formula (III), R⁵ is hydrogen; R⁹ is hydrogen; and R⁶, R⁷, and R⁸ are each independently hydrogen, —OH, or —OCH₃, wherein at least one of R⁶, R⁷, or R⁸ is not hydrogen.

In some embodiments of Formula (III), R⁵ and R⁶ together with the atoms to which they are attached form an optionally substituted C₃-C₆ cycloalkyl, optionally substituted C₃-C₆ heterocycloalkyl, optionally substituted C₆-C₈ aryl, or optionally substituted 5 to 6-membered heteroaryl, each of which is optionally substituted with 1 to 4 substituents selected from R^{d}. In some embodiments of Formula (III), R⁵ and R⁶ together with the atoms to which they are attached form an optionally substituted C₃-C₆ heterocycloalkyl. In some embodiments of Formula (III), R⁵ and R⁶ together with the atoms to which they are attached form an optionally substituted 5 to 6-membered heteroaryl. In some embodiments of Formula (III), R⁵ and R⁶ together with the atoms to which they are attached form a 5-membered heteroaryl or phenylene. In some embodiments of Formula (III), R⁵ and R⁶ together with the atoms to which they are attached form a 5-membered heteroaryl. In some embodiments of Formula (III), R⁵ and R⁶ together with the atoms to which they are attached form a phenylene.

In some embodiments of Formula (III), R⁶ and R⁷ together with the atoms to which they are attached form an optionally substituted C₃-C₆ cycloalkyl, optionally substituted C₃-C₆ heterocycloalkyl, optionally substituted C₆-C₈ aryl, or optionally substituted 5 to 6-membered heteroaryl, each of which is optionally substituted with 1 to 4 substituents selected from R^{d}. In some embodiments of Formula (III), R⁶ and R⁷ together with the atoms to which they are attached form an optionally substituted C₃-C₆ heterocycloalkyl. In some embodiments of Formula (III), R⁶ and R⁷ together with the atoms to which they are attached form an optionally substituted 5 to 6-membered heteroaryl. In some embodiments of Formula (III), R⁶ and R⁷ together with the atoms to which they are attached form a 5-membered heteroaryl or phenylene. In some embodiments of Formula (III), R⁶ and R⁷ together with the atoms to which they are attached form a 5-membered heteroaryl. In some embodiments of Formula (III), R⁶ and R⁷ together with the atoms to which they are attached form a phenylene.

In some embodiments of Formula (III), R⁷ and R⁸ together with the atoms to which they are attached form an optionally substituted C₃-C₆ cycloalkyl, optionally substituted C₃-C₆ heterocycloalkyl, optionally substituted C₆-C₈ aryl, or optionally substituted 5 to 6-membered heteroaryl, each of which is optionally substituted with 1 to 4 substituents selected from R^{d}. In some embodiments of Formula (III), R⁷ and R⁸ together with the atoms to which they are attached form an optionally substituted C₃-C₆ heterocycloalkyl. In some embodiments of Formula (III), R⁷ and R⁸ together with the atoms to which they are attached form an optionally substituted 5 to 6-membered heteroaryl. In some embodiments of Formula (III), R⁷ and R⁸ together with the atoms to which they are attached form a 5-membered heteroaryl or phenylene. In some embodiments of Formula (III), R⁷ and R⁸ together with the atoms to which they are attached form a 5-membered heteroaryl. In some embodiments of Formula (III), R⁷ and R⁸ together with the atoms to which they are attached form a phenylene.

In some embodiments of Formula (I), (II), or (III), R¹ is C₁-C₆ alkyl which is optionally substituted with 1 to 4 substituents selected from R^{e}. In some embodiment of Formula (I), (II), or (III), R¹ is —CH₃, —CH₂CH₃, —CH₂OH, —CH₂OCH₃, or —CH₂OCH₂CH₃. In some embodiments of Formula (I), (II), or (III), R¹ is hydrogen.

In some embodiments of Formula (I), (II), or (III), R² is hydrogen, C₁-C₆ alkyl, C₁-C₆ haloalkyl, or C₁-C₆ hydroxyalkyl, wherein the alkyl, haloalkyl, and hydroxyalkyl is optionally substituted with 1 to 4 substituents selected from R^{e}. In some embodiments of Formula (I), (II), or (III), R² is hydrogen or C₁-C₆ alkyl which is optionally substituted with 1 to 4 substituents selected from R^{c}. In some embodiments of Formula (I), (II), or (III), R² is C₁-C₆ alkyl. In some embodiments of Formula (I), (II), or (III), R² is —CH₃, —CH₂CH₃, —CH₂OH, —CH₂OCH₃, or —CH₂OCH₂CH₃. In some embodiments of Formula (I), (II), or (III), R² is —CH₃. In some embodiments of Formula (I), (II), or (III), R² is —CH₂OCH₃.

In some embodiments of Formula (I), (II), or (III), R¹ is hydrogen and R² is —CH₃; or R¹ and R² together with the atoms to which they are attached form a 1,3-dioxolane.

In some embodiments of Formula (I), (II), or (III), R¹ and R² together with the atoms to which they are attached for a C₃-C₅ heterocycloalkyl. In some embodiments of Formula (I), (II), or (III), R¹ and R² together with the atoms to which they are attached form a 1,3-dioxolane.

In some embodiments of Formula (I), (II), or (III), R⁴ is a straight chain, branched, saturated, or unsaturated C₄-C₂₀ alkyl. In some embodiments of Formula (I), (II), or (III), R⁴ is C₄-C₁₀ alkyl. In some embodiments of Formula (I), (II), or (III), R⁴ is unsubstituted straight chain C₄-C₈ alkyl. In some embodiments of Formula (I), (II), or (III), R⁴ is unsubstituted straight chain C₆-C₈ alkyl. In some embodiments of Formula (I), (II), or (III), R⁴ is pentyl, hexyl, heptyl, or oxatyl. In some embodiments of Formula (I), (II), or (III), R⁴ is n-pentyl, n-hexyl, or n-heptyl. In some embodiments of Formula (I), (II), or (III), R⁴ is n-pentyl. In some embodiments of Formula (I), (II), or (III), R⁴ is n-hexyl. In some embodiments of Formula (I), (II), or (III), R⁴ is n-heptyl.

In some embodiments of Formula (I), (II), or (III), Y² is —O—. In some embodiments of Formula (I), (II), or (III), Y² is —NR³—. In some embodiments of Formula (I), (II), or (III), Y² is —NH.

In some embodiments of Formula (I), (II), or (III), Z is absent, —C═C—, or C₁-C₂ alkylene which is optionally substituted with —NH₂. In some embodiments of Formula (I), (II), or (III), Z is —CH₂— or —CH(NH₂)CH₂—. In some embodiments of Formula (I), (II), or (III), Z is absent or —C═C—. In some embodiments of Formula (I), (II), or (III), Z is —O—. In some embodiments of Formula (I), (II), or (III), Z is —NH—. In some embodiments of Formula (I), (II), or (III), Z is —C═C—. In some embodiments of Formula (I), (II), or (III), Z is absent.

In some embodiments of Formula (I), (II), or (III), Y² is —O— or —NH—; and Z is absent. In some embodiments of Formula (I), (II), or (III), Y² is —O— and Z is absent. In some embodiments of Formula (I), (II), or (III), Y² is —NH—; and Z is absent.

In some embodiments of Formula (I) and (II), Y² is —O— or —NH—; and Z is —C═C—. In some embodiments of Formula (I), (II), or (III), Y² is —O— and Z is —C═C—. In some embodiments of Formula (I), (II), or (III), Y² is —NH— and Z is —C═C—.

In some embodiments described herein, each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ haloalkyl, optionally substituted C₃-C₁₀ cycloalkyl, and optionally substituted C₃-C₁₀ heterocycloalkyl, each of which is substituted with one or more R^{d}. In some embodiments, each R^{a} and R^{b} is independently selected from hydrogen, optionally substituted C₁-C₆ alkyl, optionally substituted C₁-C₆ haloalkyl, optionally substituted C₃-C₁₀ cycloalkyl, and optionally substituted C₃-C₁₀ heterocycloalkyl. In some embodiments, each R^{a} and R^{b} is independently selected from hydrogen, optionally substituted C₁-C₆ alkyl, or optionally substituted C₁-C₆ haloalkyl. In some embodiments, each R^{a} and R^{b} is independently selected from hydrogen or C₁-C₆ alkyl.

In some embodiments described herein, R^{a} and R^{b} together with the atoms to which they are attached form a C₃-C₁₀ heterocycloalkyl which is optionally substituted with one or more R^{d}. In some embodiments described herein, R^{a} and R^{b} together with the atoms to which they are attached form a C₃-C₆ heterocycloalkyl.

In some embodiments described herein, each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-C₁₀ heterocycloalkyl, optionally substituted C₆-C₁₀ aryl, and optionally substituted 5- to 10-membered heteroaryl, each of which is substituted with one or more R^{d}. In some embodiments, each R^{c} is independently selected from hydrogen, optionally substituted C₁-C₆ alkyl, optionally substituted C₁-C₆ haloalkyl, optionally substituted C₃-C₁₀ cycloalkyl, optionally substituted C₃-C₁₀ heterocycloalkyl, optionally substituted C₆-C₁₀ aryl, and optionally substituted 5- to 10-membered heteroaryl. In some embodiments, each R^{c} is independently selected from hydrogen, optionally substituted C₁-C₆ alkyl, or optionally substituted C₁-C₆ haloalkyl. In some embodiments, each R^{c} is independently selected from hydrogen or optionally substituted C₁-C₆ alkyl. In some embodiments, each R^{c} is independently selected from hydrogen, —CH₃, or —CH₂CH₃. In some embodiments, each R^{c} is independently selected from hydrogen or —CH₃. In some embodiments, each R^{c} is independently selected from optionally substituted C₃-C₁₀ cycloalkyl and optionally substituted C₃-C₁₀ heterocycloalkyl.

In some embodiments described herein, two R^{c} together with the atoms to which they are attached form a C₃-C₁₀ heterocycloalkyl which is optionally substituted with one or more R^{d}. In some embodiments described herein, two R^{c} together with the atoms to which they are attached form a C₃-C₆ heterocycloalkyl.

In some embodiments, each R^{d} is independently selected from halogen, —OH, —CH₃, —CF₃, —OCH₃, —OCF₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃. In some embodiments, each R^{d} is independently selected from halogen, —OH, —CH₃, —CF₃, —OCH₃, —OCF₃, —NH₂, —NHCH₃, and —N(CH₃)₂. In some embodiments, each R^{d} is independently selected from —C(O)OH, —C(O)OCH₃, —C(O)NH₂, —C(O)NHCH₃, —C(O)N(CH₃)₂, —NHC(O)OH, —OC(O)NH₂, and —NHC(O)CH₃. In some embodiments, each R^{d} is independently selected from halogen, —OH, —OCH₃, —CH₃, —CF₃, or —OCF₃. In some embodiments, each R^{d} is independently selected from halogen, —OH, —OCH₃, or —CH₃.

Non-limiting examples of compounds described herein, are compounds presented in Table 1 and pharmaceutically acceptable salts or solvates thereof.

TABLE 1
Representative compounds of the disclosure.

| Compound ID | Structure |
|---|---|
| 1 | 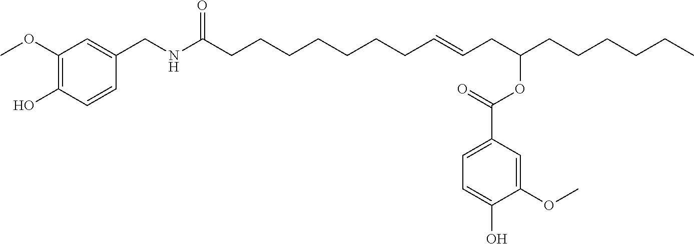 [SMILES: CCCCCCC(C/C=C/CCCCCCCC(=O)NCc1ccc(O)c(OC)c1)OC(=O)c1ccc(O)c(OC)c1] |
| 2 | 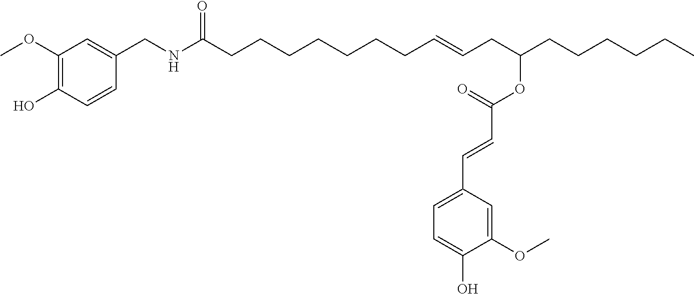 [SMILES: CCCCCCC(C/C=C/CCCCCCCC(=O)NCc1ccc(O)c(OC)c1)OC(=O)/C=C/c1ccc(O)c(OC)c1] |
| 3 | 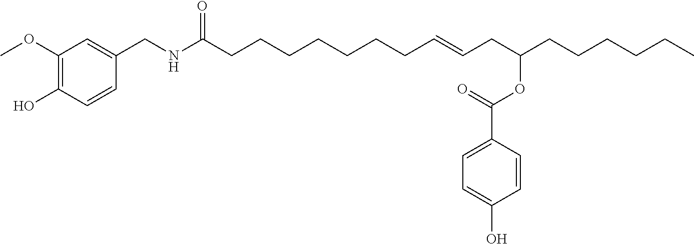 [SMILES: CCCCCCC(C/C=C/CCCCCCCC(=O)NCc1ccc(O)c(OC)c1)OC(=O)c1ccc(O)cc1] |
| 4 | 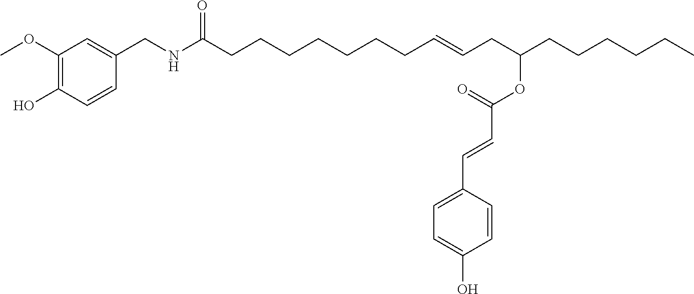 [SMILES: CCCCCCC(C/C=C/CCCCCCCC(=O)NCc1ccc(O)c(OC)c1)OC(=O)/C=C/c1ccc(O)cc1] |
| 5 | 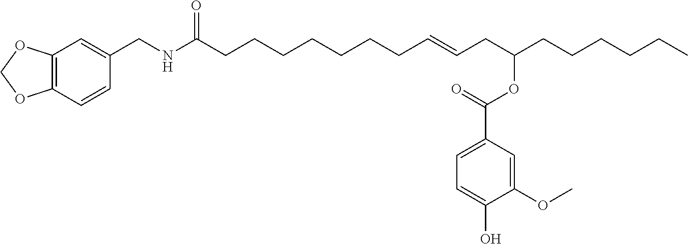 [SMILES: CCCCCCC(C/C=C/CCCCCCCC(=O)NCc1ccc2c(c1)OCO2)OC(=O)c1ccc(O)c(OC)c1] |
| 6 | 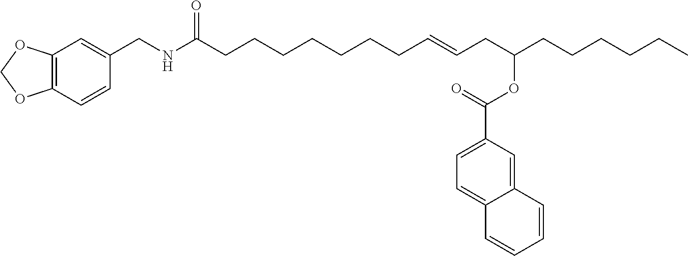 [SMILES: CCCCCCC(C/C=C/CCCCCCCC(=O)NCc1ccc2c(c1)OCO2)OC(=O)c1ccc2ccccc2c1] |
| 7 | 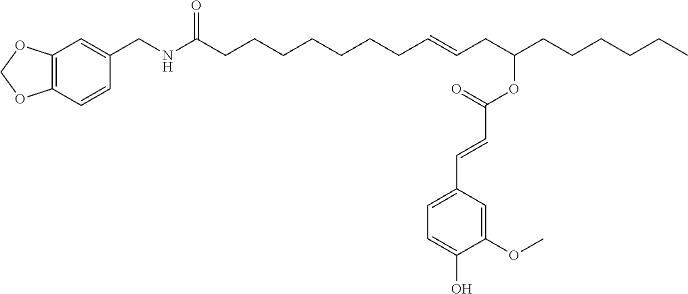 [SMILES: CCCCCCC(C/C=C/CCCCCCCC(=O)NCc1ccc2c(c1)OCO2)OC(=O)/C=C/c1ccc(O)c(OC)c1] |
| 8 | 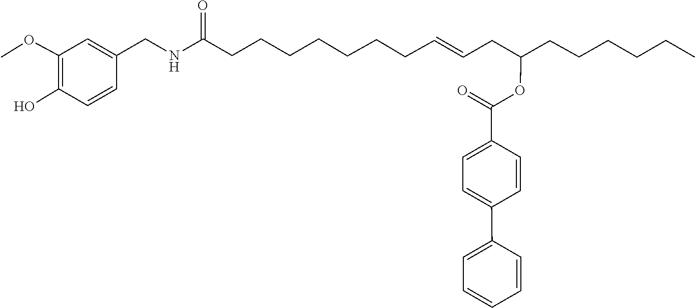 [SMILES: CCCCCCC(C/C=C/CCCCCCCC(=O)NCc1ccc(O)c(OC)c1)OC(=O)c1ccc(-c2ccccc2)cc1] |
| 9 | 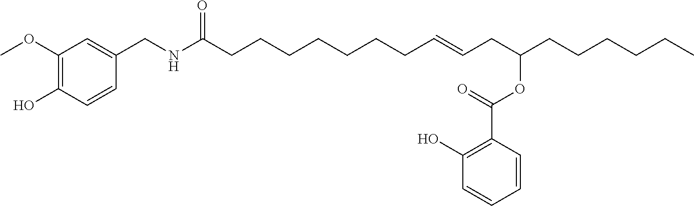 [SMILES: CCCCCCC(C/C=C/CCCCCCCC(=O)NCc1ccc(O)c(OC)c1)OC(=O)c1ccccc1O] |
| 10 | 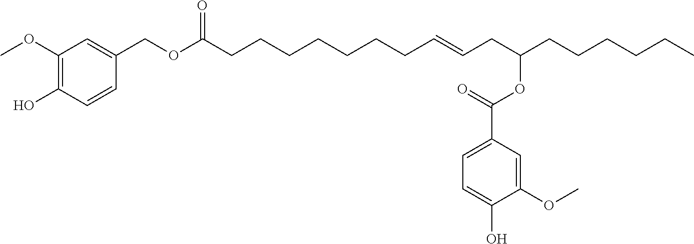 [SMILES: CCCCCCC(C/C=C/CCCCCCCC(=O)OCc1ccc(O)c(OC)c1)OC(=O)c1ccc(O)c(OC)c1] |
| 11 | 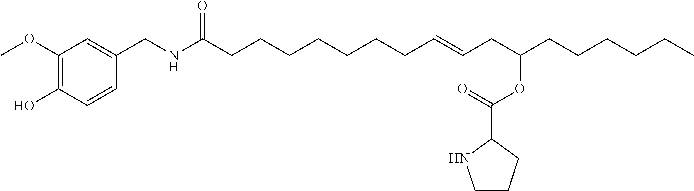 [SMILES: CCCCCCC(C/C=C/CCCCCCCC(=O)NCc1ccc(O)c(OC)c1)OC(=O)C1CCCN1] |
| 12 | 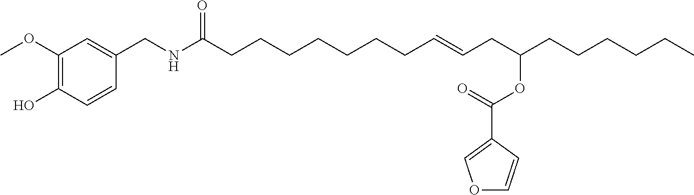 [SMILES: CCCCCCC(C/C=C/CCCCCCCC(=O)NCc1ccc(O)c(OC)c1)OC(=O)c1ccoc1] |
| 13 | 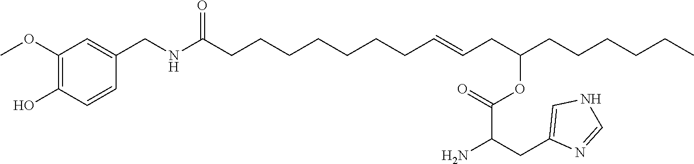 [SMILES: CCCCCCC(C/C=C/CCCCCCCC(=O)NCc1ccc(O)c(OC)c1)OC(=O)C(N)Cc1c[nH]cn1] |
| 14 | 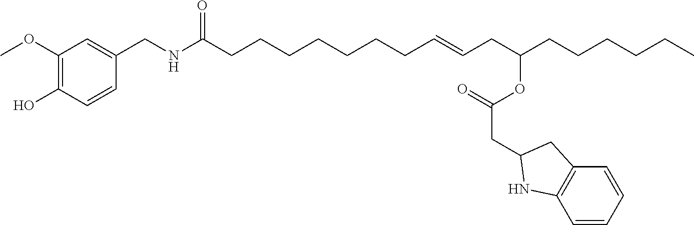 [SMILES: CCCCCCC(C/C=C/CCCCCCCC(=O)NCc1ccc(O)c(OC)c1)OC(=O)CC1Cc2ccccc2N1] |
| 15 | 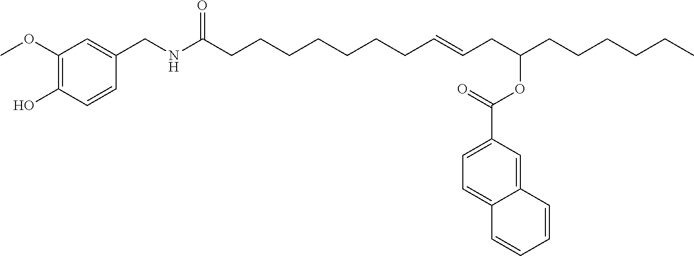 [SMILES: CCCCCCC(C/C=C/CCCCCCCC(=O)NCc1ccc(O)c(OC)c1)OC(=O)c1ccc2ccccc2c1] |
| 16 | 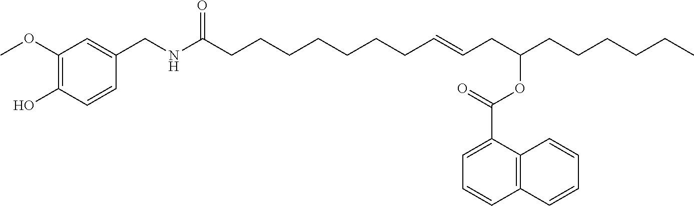 [SMILES: CCCCCCC(C/C=C/CCCCCCCC(=O)NCc1ccc(O)c(OC)c1)OC(=O)c1cccc2ccccc12] |
| 17 | 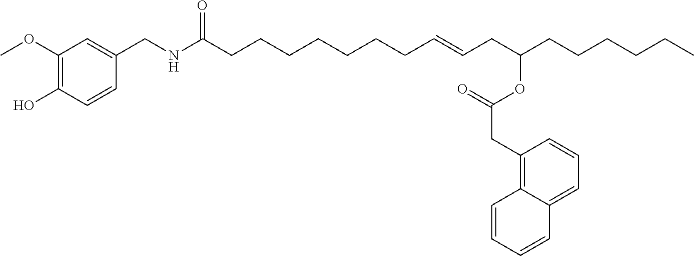 [SMILES: CCCCCCC(C/C=C/CCCCCCCC(=O)NCc1ccc(O)c(OC)c1)OC(=O)Cc1cccc2ccccc12] |
| 18 | 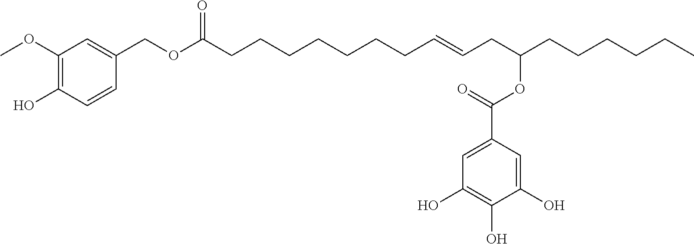 [SMILES: CCCCCCC(C/C=C/CCCCCCCC(=O)OCc1ccc(O)c(OC)c1)OC(=O)c1cc(O)c(O)c(O)c1] |
| 19 | 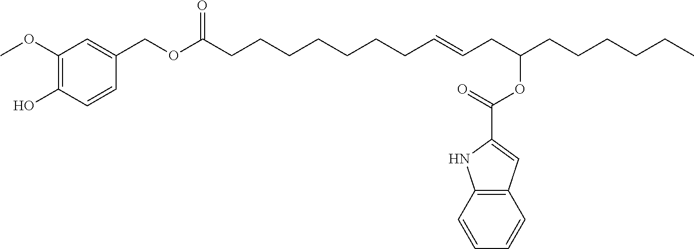 [SMILES: CCCCCCC(C/C=C/CCCCCCCC(=O)OCc1ccc(O)c(OC)c1)OC(=O)c1cc2ccccc2[nH]1] |
| 20 | 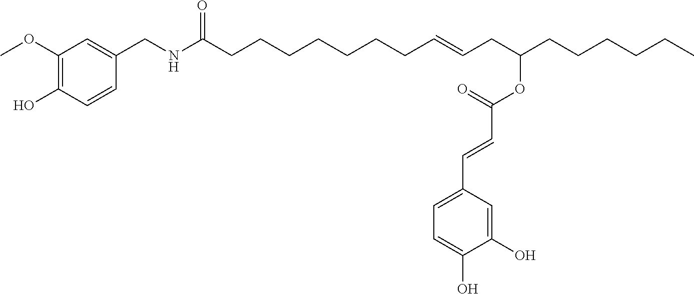 [SMILES: CCCCCCC(C/C=C/CCCCCCCC(=O)NCc1ccc(O)c(OC)c1)OC(=O)/C=C/c1ccc(O)c(O)c1] |
| 21 | 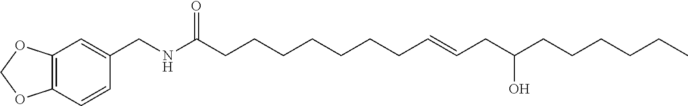 [SMILES: CCCCCCC(O)C/C=C/CCCCCCCC(=O)NCc1ccc2c(c1)OCO2] |
| 22 Rinvanil | 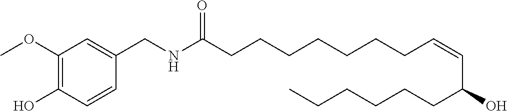 [SMILES: CCCCCC[C@H](O)/C=C\CCCCCCCC(=O)NCc1ccc(O)c(OC)c1] |
| 23 | 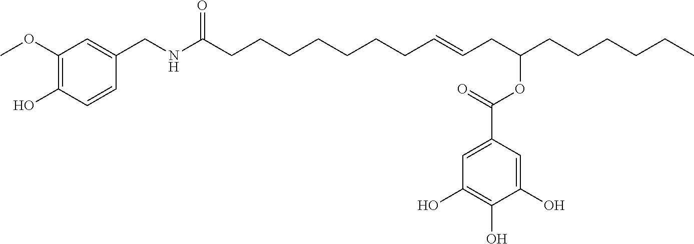 [SMILES: CCCCCCC(C/C=C/CCCCCCCC(=O)NCc1ccc(O)c(OC)c1)OC(=O)c1cc(O)c(O)c(O)c1] |
| 24 | 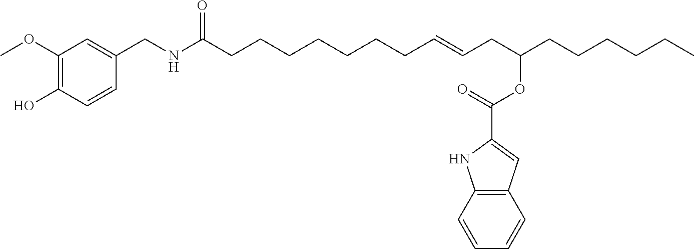 [SMILES: CCCCCCC(C/C=C/CCCCCCCC(=O)NCc1ccc(O)c(OC)c1)OC(=O)c1cc2ccccc2[nH]1] |

Included in the present disclosure are salts, particularly pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

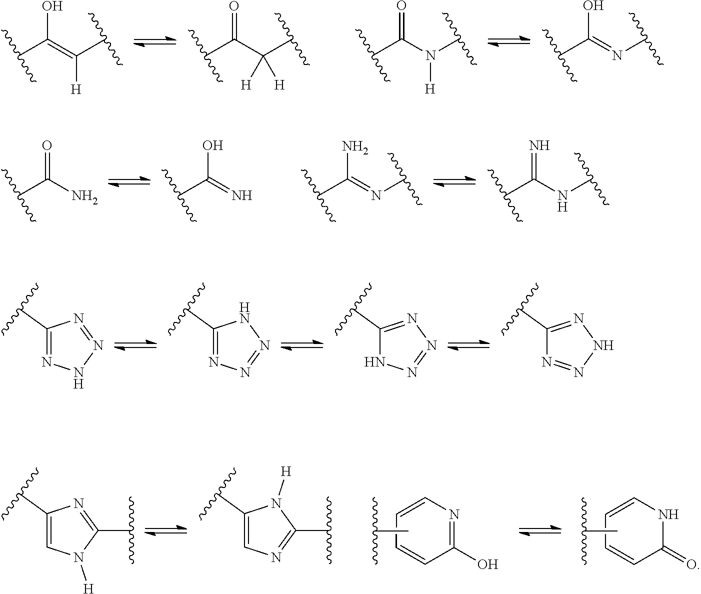

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N ¹⁶N, ¹⁶O, ¹⁷O, ¹⁴F, ¹⁵F, ¹⁶F, ¹⁷F, ¹⁸F, 33S, 34S, 35S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% of a total number of hydrogen and deuterium. In some embodiments, one or more of the substituents disclosed herein comprise deuterium at a percentage higher than the natural abundance of deuterium. In some embodiments, one or more hydrogens are replaced with one or more deuteriums in one or more of the substituents disclosed herein.

The compounds described herein may in some cases exist as diastereomers, enantiomers, atropisomers, or other stereoisomeric forms. Where absolute stereochemistry is not specified, the compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser 's Reagents for Organic Synthesis (1994); and L. Paquette, ed., Encyclopedia of Reagents for Organic Synthesis (1995).

### Pharmaceutical Compositions

Provided herein, in certain embodiments, are compositions comprising a therapeutically effective amount of any compound or salt or solvate of Formula (I), (II), and (III), (also referred to herein as “a pharmaceutical agent”).

Pharmaceutical compositions may be formulated using one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical composition comprising, for example, a pharmaceutical agent and a pharmaceutically acceptable carrier or excipient. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule, granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable excipient can contain physiologically acceptable agents that act, for example, to stabilize, increase solubility or to increase the absorption of a compound such as a pharmaceutical agent. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self-emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, pastes for application to the tongue; absorption through the oral mucosa, e.g., sublingually; anally, rectally or vaginally, for example, as a pessary, cream or foam; parenterally, including intramuscularly, intravenously, subcutaneously or intrathecally as, for example, a sterile solution or suspension; nasally; intraperitoneally; subcutaneously; transdermally, for example, as a patch applied to the skin; and topically, for example, as a cream, ointment or spray applied to the skin, or as an eye drop. The compound may also be formulated for inhalation. In certain embodiments, a compound may be simply dissolved or suspended in sterile water.

A pharmaceutical composition may be a sterile aqueous or non-aqueous solution, suspension or emulsion, e.g., a microemulsion. The excipients described herein are examples and are in no way limiting. An effective amount or therapeutically effective amount refers to an amount of the one or more pharmaceutical agents administered to a subject, either as a single dose or as part of a series of doses, which is effective to produce a desired therapeutic effect.

Subjects may generally be monitored for therapeutic effectiveness using assays and methods suitable for the condition being treated, which assays will be familiar to those having ordinary skill in the art and are described herein. Pharmacokinetics of a pharmaceutical agent, or one or more metabolites thereof, that is administered to a subject may be monitored by determining the level of the pharmaceutical agent or metabolite in a biological fluid, for example, in the blood, blood fraction, e.g., serum, and/or in the urine, and/or other biological sample or biological tissue from the subject. Any method practiced in the art and described herein to detect the agent may be used to measure the level of the pharmaceutical agent or metabolite during a treatment course.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

Pharmaceutical compositions comprising a pharmaceutical agent can be formulated in a manner appropriate for the delivery method by using techniques routinely practiced in the art. The composition may be in the form of a solid, e.g., tablet, capsule, semi-solid, e.g., gel, liquid, or gas, e.g., aerosol. In other embodiments, the pharmaceutical composition is administered as a bolus infusion.

Pharmaceutical acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21^{st} Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the pharmaceutical agent(s) of the composition upon administration. In other embodiments, the pharmaceutical agent may be encapsulated within liposomes using technology known and practiced in the art. In certain particular embodiments, a pharmaceutical agent is not formulated within liposomes for application to a stent that is used for treating highly, though not totally, occluded arteries. Pharmaceutical compositions may be formulated for any appropriate manner of administration described herein and, in the art.

A pharmaceutical composition, e.g., for oral administration or for injection, infusion, subcutaneous delivery, intramuscular delivery, intraperitoneal delivery or other method, may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the pharmaceutical agents described herein can be used alone or in combination with appropriate additives to make tablets, powders, granules or capsules, and if desired, with diluents, buffering agents, moistening agents, preservatives, coloring agents, and flavoring agents. The pharmaceutical agents may be formulated with a buffering agent to provide for protection of the compound from low pH of the gastric environment and/or an enteric coating. A pharmaceutical agent included in a pharmaceutical composition may be formulated for oral delivery with a flavoring agent, e.g., in a liquid, solid or semi-solid formulation and/or with an enteric coating.

A pharmaceutical composition comprising any one of the pharmaceutical agents described herein may be formulated for sustained or slow release, also called timed release or controlled release. Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible and may also be biodegradable; preferably the formulation provides a relatively constant level of active component release. The amount of pharmaceutical agent contained within a sustained release formulation depends upon the site of implantation, the rate and expected duration of release, and the nature of the condition, disease or disorder to be treated or prevented.

In certain embodiments, the pharmaceutical compositions comprising a pharmaceutical agent are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered topically or injected directly into the skin adjacent to or within the area to be treated, e.g., intradermally or subcutaneously. The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

Pharmaceutical compositions comprising a pharmaceutical agent can be formulated as emulsions for topical application. An emulsion contains one liquid distributed in the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions, patches, bandages and articles. These systems are semisolid emulsions, micro-emulsions, or foam emulsion systems.

In some embodiments, the pharmaceutical agent described herein can be formulated as in inhalant. Inhaled methods can deliver medication directly to the airway. The pharmaceutical agent can be formulated as aerosols, microspheres, liposomes, or nanoparticles. The pharmaceutical agent can be formulated with solvents, gases, nitrates, or any combinations thereof. Compositions described herein are optionally formulated for delivery as a liquid aerosol or inhalable dry powder. Liquid aerosol formulations are optionally nebulized predominantly into particle sizes that can be delivered to the terminal and respiratory bronchioles. Liquid aerosol and inhalable dry powder formulations are preferably delivered throughout the endobronchial tree to the terminal bronchioles and eventually to the parenchymal tissue.

Aerosolized formulations described herein are optionally delivered using an aerosol forming device, such as a jet, vibrating porous plate or ultrasonic nebulizer, preferably selected to allow the formation of aerosol particles having with a mass medium average diameter predominantly between 1 to 5 p. Further, the formulation preferably has balanced osmolarity ionic strength and chloride concentration, and the smallest aerosolizable volume able to deliver effective dose of the pharmaceutical agent. Additionally, the aerosolized formulation preferably does not impair negatively the functionality of the airways and does not cause undesirable side effects.

Aerosolization devices suitable for administration of aerosol formulations described herein include, for example, jet, vibrating porous plate, ultrasonic nebulizers and energized dry powder inhalers, that are able to nebulize the formulation into aerosol particle size predominantly in the size range from 1-5 p. Predominantly in this application means that at least 70% but preferably more than 90% of all generated aerosol particles are within 1-5 p range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb™ and AeroDose™ vibrating porous plate nebulizers (AeroGen, Inc., Sunnyvale, California), Sidestream® nebulizers (Medic-Aid Ltd., West Sussex, England), Pari LC® and Pari LC Star@jet nebulizers (Pari Respiratory Equipment, Inc., Richmond, Virginia), and Aerosonic™ (DeVilbiss Medizinische Produkte (Deutschland) GmbH, Heiden, Germany) and UltraAire® (Omron Healthcare, Inc., Vernon Hills, Illinois) ultrasonic nebulizers.

In some embodiments, the pharmaceutical agent(s) can be formulated with oleaginous bases or ointments to form a semisolid composition with a desired shape. In addition to the pharmaceutical agent, these semisolid compositions can contain dissolved and/or suspended bactericidal agents, preservatives and/or a buffer system. A petrolatum component that may be included may be any paraffin ranging in viscosity from mineral oil that incorporates isobutylene, colloidal silica, or stearate salts to paraffin waxes. Absorption bases can be used with an oleaginous system. Additives may include cholesterol, lanolin (lanolin derivatives, beeswax, fatty alcohols, wool wax alcohols, low HLB (hydrophobellipophobe balance) emulsifiers, and assorted ionic and nonionic surfactants, singularly or in combination.

Controlled or sustained release transdermal or topical formulations can be achieved by the addition of time-release additives, such as polymeric structures, matrices, that are available in the art. For example, the compositions may be administered through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the compound.

### Methods of Treatment

In some embodiments, the present disclosure provides for compounds that function as modulators of transient receptor potential vanilloid 1 (TRPV1). In some embodiments, the compounds disclosed herein that function as agonists of TRPV1.

In another aspect, provided herein is a method of treating a disease or condition mediated by transient receptor potential vanilloid 1 (TRPV1) activity in a subject in need thereof, comprising administering to the subject a compound of Formula (I), (II), or (III), or a pharmaceutically acceptable salt or solvate thereof.

In another aspect, provided herein is a method of treating a metabolic condition in a subject in need thereof, the method comprising administering to the subject a therapeutically effective amount of a compound of Formula (I), (II), or (III), or a pharmaceutically acceptable salt or solvate thereof.

In some embodiments, the metabolic condition is obesity.

In another aspect, provided herein is a method of treating obesity in a subject in need thereof, the method comprising administering to the subject a therapeutically effective amount of a compound of Formula (I), (II), or (III), or a pharmaceutically acceptable salt or solvate thereof.

In another aspect, provided herein is a method of treating pain in a subject in need thereof, the method comprising administering to the subject a therapeutically effective amount of a compound of Formula (I), (II), or (III), or a pharmaceutically acceptable salt or solvate thereof.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-recloseable containers. Alternatively, multiple-dose recloseable containers can be used, in which case it is typical to include a preservative in the composition. By way of example only, formulations for parenteral injection may be presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Compound Synthesis

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser 's Reagents for Organic Synthesis (1994); and L. Paquette, ed., Encyclopedia of Reagents for Organic Synthesis (1995).

### General Synthetic Methods

### Scheme 1.

General enzymatic method for the production of amides and esters mediated by commercially available lipases in organic solvents.

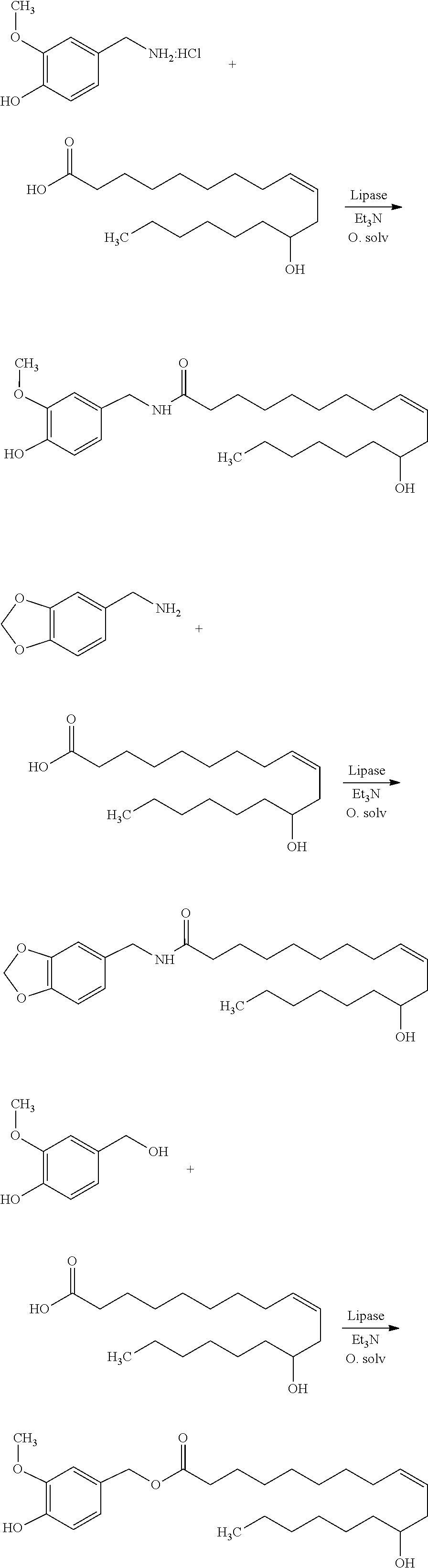

### Scheme 2.

General chemical procedure for Mitsunobu reaction to transform the corresponding amide.

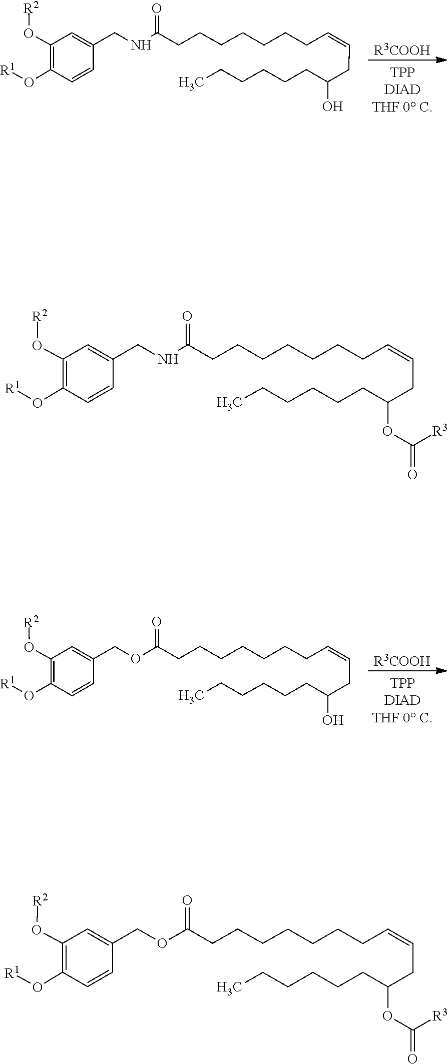

### Example 1. Synthesis of Compound 1

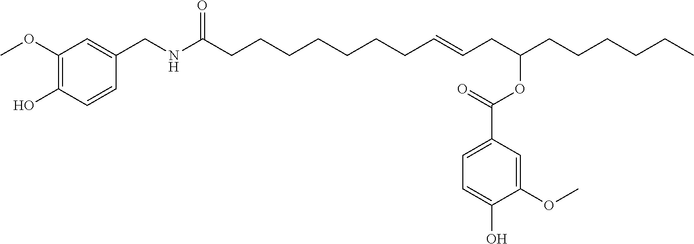 [SMILES: CCCCCCC(C/C=C/CCCCCCCC(=O)NCc1ccc(O)c(OC)c1)OC(=O)c1ccc(O)c(OC)c1]

To a suspension of vanillylamine:HCl (50 mmol) in 2-methyl-2-butanol (250 ml), Et3N (0.3 M, 6 eq.) was added. The mixture was incubated for 30 min at 37° C. and 250 rpm. Amide synthesis was started with the addition of ricinoleic acid (50 mmol) and Liposyme 435 (10 gr). After 48 h, the mixture was filtered over Celite and the solvent evaporated under vacuum. Rinvanil was purified through chromatography column (silica gel 230-400 mesh, 7:3 hexane-EtOAc) and obtained as a yellowish oil.

¹H NMR (400 MHz, CDCl₃) δ (ppm): 6.83-6.70 (m), 6.30-6.28 (br s), 5.53-5.38 (m), 4.31-4.30 (m), 3.62-3.59 (s), 2.30-1.99 (m), 1.62-1.45 (m), 1.28-1.17 (br m), 0.88-0.86 (t). ¹³C NMR (CDCl₃) δ (ppm): 173.4592, 146.9359, 145.2300, 133.1171, 130.2431, 125.3629, 120.6814, 114.6311, 110.8860, 71.6030, 55.8907, 43.5102, 36.7941, 35.6798, 35.3208, 31.8870, 29.5810, 29.4646, 29.2421, 29.2146, 29.1384, 27.3441, 25.7828, 25.7583, 22.6751, 14.1538.

To a cooled (0° C.) solution of rinvanil (5 mM) and vanillic acid (5 mM) in dry THF, triphenylphosphine (TPP, 5 mM, 1 eq.) was added. After solvation, diisopropyl azodicarboxylate (DIAD, 5 mM, 1 eq.) was added dropwise. The reaction was stirred at room temperature for 72 h and then the solvent was evaporated under vacuum. The product was purified by chromatography column (silica gel 230-400 mesh, 8:2 hexane-EtOAc) to obtain Compound 1 as a white solid.

¹H NMR (CDCl₃) δ (ppm): 7.68-7.26 (m), 3.95-3.79 (m), 1.86 (br s), 1.31-1.25 (br m), 0.88 (t). ¹³C NMR (CDCl₃) δ (ppm): 133.0258, 132.1584, 132.0060, 131.9575, 131.9467, 128.5785, 128.4587.

### Example 2. Synthesis of Compound 2

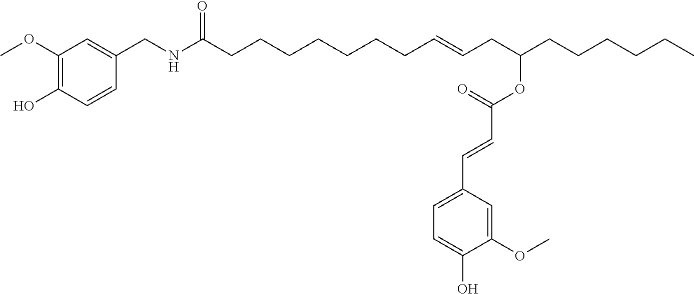 [SMILES: CCCCCCC(C/C=C/CCCCCCCC(=O)NCc1ccc(O)c(OC)c1)OC(=O)/C=C/c1ccc(O)c(OC)c1]

To a cooled (0° C.) solution of rinvanil (5 mM) and ferulic acid (5 mM) in dry THF, triphenylphosphine (TPP, 5 mM, 1 eq.) was added. After solvation, diisopropyl azodicarboxylate (DIAD, 5 mM, 1 eq.) was added dropwise. The reaction was stirred at room temperature for 72 h and then the solvent was evaporated under vacuum. The product was purified by chromatography column (silica gel 230-400 mesh, 8.5:1.5 hexane-EtOAc) to obtain Compound 2 as a yellowish solid.

¹H NMR (400 MHz, CDCl₃) δ (ppm): 7.80-7.75 (m), 7.15-6.94 (m), 6.39-6.35 (m), 5.03-4.93 (m), 4.15-3.88 (m), 2.05 (br s), 1.28-1.24 (br in), 0.88 (t). ¹³C NM/R (CDCl₃) δ (ppm): 171.25, 162.98, 156.40, 148.91, 147.11, 146.89, 126.39, 124.00, 123.50, 114.91, 114.05, 109.69, 70.15, 60.44, 56.02, 21.98, 21.08, 14.21.

### Example 3. Synthesis of Compound 12

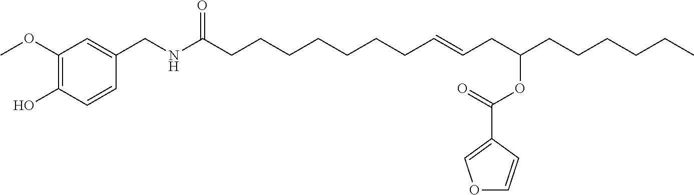 [SMILES: CCCCCCC(C/C=C/CCCCCCCC(=O)NCc1ccc(O)c(OC)c1)OC(=O)c1ccoc1]

To a cooled (0° C.) solution of rinvanil (5 mM) and 3-furoic acid (5 mM) in dry THF, triphenylphosphine (TPP, 5 mM, 1 eq.) was added. After solvation, diisopropyl azodicarboxylate (DIAD, 5 mM, 1 eq.) was added dropwise. The reaction was stirred at room temperature for 72 h and then the solvent was evaporated under vacuum. The product was purified by chromatography column (silica gel 230-400 mesh, 7.5:2.5 hexane-EtOAc) to obtain Compound 12 as a transparent resin.

¹H NMR (CDCl₃) δ (ppm): 7.98-7.97 (m), 7.74-7.40 (m), 6.88-6.73 (m), 5.75-5.69 (m), 5.47-5.35 (m), 5.07-5.01 (m), 4.36-4.34 (m), 4.14-4.09 (m), 3.90 (s), 2.41-2.33 (m), 2.21-2.17 (m), 2.05-2.00 (m), 1.66-1.62 (m), 1.28-1.25 (m), 0.88-0.86 (t). ¹³C NMR (CDCl₃) δ (ppm): 172.9368, 162.9688, 147.5206, 146.6605, 145.1146, 143.6151, 132.7556, 130.3858, 124.1300, 120.8049, 119.8301, 114.3722, 110.9892, 55.9328, 43.5364, 36.8223, 33.6801, 31.7287, 31.6487, 29.2269, 29.2080, 29.1734, 25.7564, 25.7366, 22.5923, 14.0823.

### Example 4. Synthesis of Compound 17

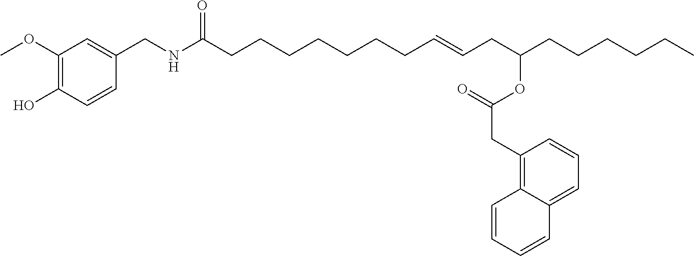 [SMILES: CCCCCCC(C/C=C/CCCCCCCC(=O)NCc1ccc(O)c(OC)c1)OC(=O)Cc1cccc2ccccc12]

To a cooled (0° C.) solution of rinvanil (5 mM) and 1-Naphthaleneacetic acid (5 mM) in dry THF, triphenylphosphine (TPP, 5 mM, 1 eq.) was added. After solvation, diisopropyl azodicarboxylate (DIAD, 5 mM, 1 eq.) was added dropwise. The reaction was stirred at room temperature for 72 h and then the solvent was evaporated under vacuum. The product was purified by chromatography column (silica gel 230-400 mesh, 8:2 hexane-EtOAc) to obtain Compound 17 as a white solid.

¹H NMR (CDCl₃) δ (ppm): 7.98-7.85 (m), 7.78-7.40 (m), 6.88-6.73 (m), 5.68-5.63 (m), 5.41-5.22 (m), 4.89-4.83 (m), 4.33-4.32 (m), 4.03-3.99 (s), 2.28-2.17 (m), 2.05-1.99 (m), 1.66-1.61 (m), 1.47-1.26 (m), 0.88-0.82 (t). ¹³C NMR (CDCl₃) δ (ppm): 172.8662, 171.4146, 146.6731, 145.1003, 133.8189, 132.6168, 132.2168, 130.2308, 130.0866, 129.8489, 127.9800, 127.8501, 126.7198, 125.9189, 125.4503, 124.1503, 123.9089, 123.7998, 123.5986, 120.9867, 110.6861, 55.9347, 43.5061, 39.5883, 36.7944, 33.5456, 31.6614, 31.6419, 29.7180, 29.4780, 29.0397, 29.0037, 28.5530, 25.7338, 25.7033, 22.5136, 14.0789.

### Example 5. Synthesis of Compound 21

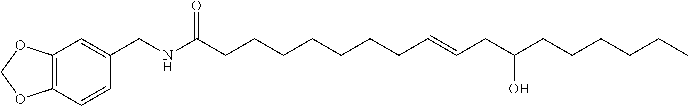 [SMILES: CCCCCCC(O)C/C=C/CCCCCCCC(=O)NCc1ccc2c(c1)OCO2]

To a stirred solution of piperonylamine (0.15 M) and ricinoleic acid (0.15 M) in dry 2M2B (45° C., 100 ml), Lipozyme 435 (5 gr) was added. After 48 h, the enzyme was filtrated through Celite and the solvent was evaporated under vacuum. The product was purified by chromatography column (silica gel 230-400 mesh, 1:1 hexane-EtOAc) to obtain Compound 21 as a white powder.

¹H NMR (400 MHz, CDCl₃) δ (ppm): 6.77-6.71 (m), 6.01 (s), 5.73-5.38 (m), 4.34-4.33 (m), 3.62-3.59 (m), 2.22-2.03 (m), 1.65-1.43 (m), 0.89-0.85 (t). ¹³C NMR (CDCl₃) δ (ppm): 172.9208, 147.9139, 146.9591, 133.4275, 132.2028, 125.2460, 121.1117, 108.4409, 108.3077, 101.0816, 71.5029, 43.4085, 36.7636, 35.8628, 35.3735, 31.8534, 29.5452, 29.4350, 29.2418, 29.1394, 27.3357, 25.7476, 24.6419, 22.6419, 14.1192.

### Example 6. Synthesis of Compound 22

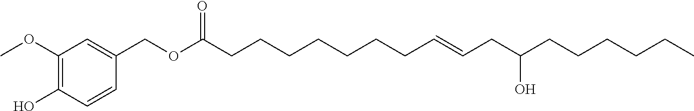 [SMILES: CCCCCCC(O)C/C=C/CCCCCCCC(=O)OCc1ccc(O)c(OC)c1]

To a suspension of vanillyl alcohol (50 mmol) in acetone (250 ml, 45° C.) and ricinoleic acid (50 mmol), Liposyme 435 (10 gr) was added. After 72 h, the mixture was filtered over Celite and the solvent evaporated under vacuum. Rinvanol (Compound 22) was purified through chromatography column (silica gel 230-400 mesh, 7:3 hexane-EtOAc) to obtain Compound 22 as a transparent oil.

¹H NMR (400 MHz, CDCl₃) δ (ppm): 6.89-6.86 (m), 5.56-5.50 (m), 5.41-5.38 (m), 5.02 (s), 3.90-3.85 (m), 3.66-3.58 (m), 2.34-2.20 (m), 2.10-1.95 (m), 1.70-1.42 (m), 0.89-0.86 (t). ¹³C NMR (CDCl₃) δ (ppm): 173.81, 146.47, 145.75, 133.45, 128.00, 125.29, 122.04, 114.36, 111.26, 71.53, 66.31, 55.94, 36.84, 35.35, 34.18, 31.65, 29.58, 29.34, 29.14, 27.06, 25.75, 22.64, 14.1.

### Example 7. Synthesis of Compound 20

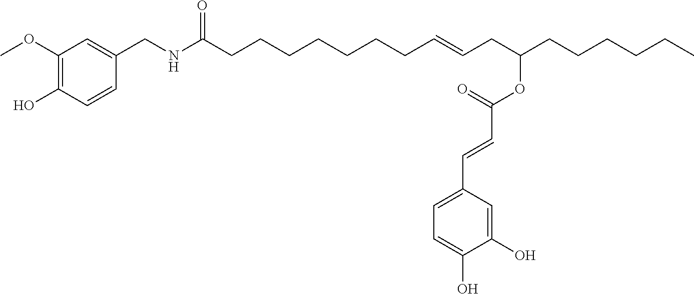 [SMILES: CCCCCCC(C/C=C/CCCCCCCC(=O)NCc1ccc(O)c(OC)c1)OC(=O)/C=C/c1ccc(O)c(O)c1]

To a cooled (0° C.) solution of rinvanil (5 mM) and caffeic acid (5 mM) in dry THF, triphenylphosphine (TPP, 5 mM, 1 eq.) was added. After solvation, diisopropyl azodicarboxylate (DIAD, 5 mM, 1 eq.) was added dropwise. After stirring at room temperature for 72 h, the solvent was evaporated under vacuum. The product was purified by chromatography column (silica gel 230-400 mesh, 8:2 hexane-EtOAc) to obtain Compound 20 as a yellowish resin.

¹H NMR (400 MHz, CDCl₃) δ (ppm): 7.54 (d), 7.50 (d), 6.93-6.74 (m), 6.18 (d), 5.56-5.38 (m), 4.36-4.34 (m), 3.87 (s), 3.62-3.58 (m), 2.23-1.95 (m), 1.65-1.20 (br m), 0.88-0.86 (t). ¹³C NMR (CDCl₃) δ (ppm): 145.1211, 133.4518, 130.3054, 125.2352, 120.8216, 114.4057, 110.7220, 71.5706, 55.9539, 43.9777, 43.5982, 36.8206, 35.3443, 31.8546, 29.6384, 29.5572, 29.3385, 29.1671, 27.1430, 25.7499, 22.6437, 14.1181.

### Example 8. Synthesis of Compound 23

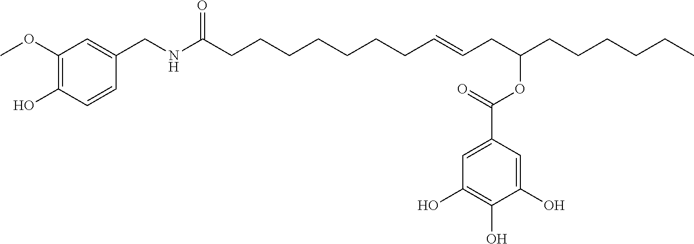 [SMILES: CCCCCCC(C/C=C/CCCCCCCC(=O)NCc1ccc(O)c(OC)c1)OC(=O)c1cc(O)c(O)c(O)c1]

To a cooled (0° C.) solution of rinvanil (5 mM) and gallic acid (5 mM) in dry THF, triphenylphosphine (TPP, 5 mM, 1 eq.) was added. After solvation, diisopropyl azodicarboxylate (DIAD, 5 mM, 1 eq.) was added dropwise. After stirring at room temperature for 72 h, the solvent was evaporated under vacuum. The product was purified by chromatography column (silica gel 230-400 mesh, 8:2 hexane-EtOAc) to obtain Compound 23 as a brownish resin.

¹H NMR (400 MHz, CDCl₃) δ (ppm): 7.72-7.21 (m), 6.86-6.73 (m), 6.11-6.10 (br s), 5.74-5.33 (m), 4.36-4.34 (m), 3.85-3.84 (s), 2.37-2.18 (m), 1.63-1.55 (m), 1.28-1.15 (m), 0.87-0.84 (t). ¹³C NMR (CDCl₃) δ (ppm): 166.2688, 146.7542, 145.2157, 144.8310, 132.8147, 131.1076, 124.2972, 120.8764, 114.4212, 110.6858, 109.7688, 60.4420, 55.9348, 43.7705, 36.7349, 33.8407, 31.9809, 31.7555, 29.2352, 29.1565, 29.1387, 28.9975, 27.3475, 25.8106, 25.5833, 22.6812, 21.0882, 14.2156, 14.0944.

### Example 9. Synthesis of Compound 24

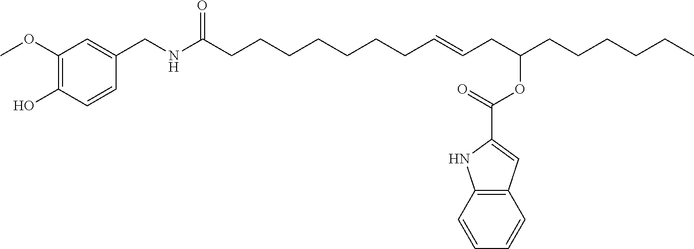 [SMILES: CCCCCCC(C/C=C/CCCCCCCC(=O)NCc1ccc(O)c(OC)c1)OC(=O)c1cc2ccccc2[nH]1]

Rinvanil (5 mM), indole-2-carboxylic acid (5 mM) and TPP (5 mM) were added to cool (0° C.) and dry THF (5 ml). After homogeneity, DIAD (5 mM) was added dropwise. The reaction was stirred at room temperature for 72 h and then the solvent was evaporated under vacuum. The product was purified by chromatography column (silica gel 230-400 mesh, 8:2 hexane-EtOAc) to obtain Compound 24 as a whitish resin.

¹H NMR (400 MHz, CDCl₃) δ (ppm): 7.68-7.11 (m), 6.87-6.74 (m), 5.68-5.10 (m), 4.36-4.34 (m), 3.85-3.84 (s), 2.47-2.02 (m), 1.69-1.56 (m), 1.33-1.25 (m), 0.88-0.84 (t). ¹³C NMR (CDCl₃) δ (ppm): 173.0745, 161.8419, 146.7039, 145.1219, 136.8602, 132.9999, 130.8385, 127.7691, 127.4827, 125.2153, 123.9548, 122.5048, 120.8192, 114.3822, 111.9483, 110.7061, 108.4918, 55.9365, 43.5545, 36.8094, 33.7897, 31.7421, 29.1033, 29.1634, 29.0449, 27.3362, 25.4360, 22.5980, 14.0757.

### Example B-1: 3T3-L1 Preadipocytes Differentiation

The 3T3-L1 fibroblasts are maintained in DMEM supplemented with 10% of fetal bovine serum, glutamine, penicillin, and streptomycin at 37° C. in an atmosphere of 5% CO₂. To induce adipocyte differentiation, the 3T3-L1 cells are allowed to grow to confluence during culture with a differentiation medium that contains 3-isobutyl-1-methylxanthine, dexamethasone, and insulin. After 48 h of exposure to the differentiation medium, the cells are maintained for an additional 5-7 days in supplemented DMEM. Fully differentiated 3T3-L1 adipocytes in serum-free medium are treated with compounds disclosed herein dissolved in DMSO for 24 h.

### Example B-2: Total Lipids and Triglyceride Accumulation in 3T3-L1 Cells

The accumulation of total lipid content is measured by staining with oil red O dye for 15 min. The stained oil droplets in the cell monolayer are dissolved with isopropanol, followed by measuring the absorbance at 520 nm in a microplate spectrophotometer.

### Example B-3: Quantification of Intracellular Triglyceride Content

To evaluate the triglyceride content, the extraction and quantification of total protein is first performed. Supplemented RIPA buffer solution is added to each well and the plates are kept on ice for 10 min. The cells were then scraped off with the help of a micropipette tip. The cell suspension is collected and kept on ice for 1 h, vortexing every 30 min. Subsequently, the samples were centrifuged, the supernatant containing the proteins is collected, and aliquots of each were made. For the quantification of triglyceride content, the cells are lysed in a lysis buffer and the cellular content of triglyceride is measured using a TG colorimetric assay kit in which triglycerides are converted into fatty acids and glycerol at the time of adding the lipase enzyme. The free glycerol is oxidized and generates a color, which was quantified at 570 nm in the spectrophotometer. The content of TG concentration is expressed on nm/mg of protein.

### Example B-4: Diet-Induced Obese Mice

Four-week-old, male C57BL/6J mice were obtained and housed between four to five per cage at 24±0.5° C. on a 12-h day/night cycle. Mice received standard diet (SD), mouse chow for 2 weeks on quarantine and then they were moved to a new environment where there were randomly assigned to n groups. They were fed for 12 weeks with their respective diet. After this period, they were given their respective treatment orally with a gauge for 4 weeks. All mice were fasted overnight before being euthanized with a lethal injection of sodium pentobarbital.

### Example B-5: Biochemical Assays and Fat Pad Collection

Blood samples were collected from each animal by cardiac puncture. The plasma and serum was separated and stored at −20° C. Fasting glucose concentration, total cholesterol (TC), triglycerides (TG), high-density lipoproteins (HDL), low-density lipoproteins (LDL), very low-density lipoproteins and (VLDL), were measured by automated enzymatic methods on a Sincron-7 analyzer. Finally, two types of white adipose tissues (visceral and subcutaneous) were dissected and weighed.

### Example B-6: Nano-Differential Scanning Calorimetry (NanoDSC)

Thermal stability of TRPV1 in the presence of test compounds was evaluated using nano-differential scanning calorimetry (NanoDSC).

Purified rat TRPV1 protein was prepared at a concentration of approximately 0.8 mg/mL in a buffer comprising 150 mM NaCl, 20 mM HEPES, 0.5 mM TCEP, 0.5 mM dodecyl maltoside (DDM), and lipid supplement, adjusted to pH 7.4. Test compounds were added to the protein solution at an approximate molar ratio of 10:1 (compound:protein). Compounds were prepared as stock solutions in dimethyl sulfoxide (DMSO), and corresponding volumes were added to the protein samples such that the final DMSO concentration did not exceed approximately 0.1% (v/v).

Protein-compound mixtures were incubated at room temperature for approximately 30 minutes prior to calorimetric analysis. Samples were then loaded into a nano-differential scanning calorimeter and equilibrated at approximately 2° C. prior to data acquisition. Thermal denaturation profiles were obtained by heating samples from approximately 2° C. to approximately 95° C. at a constant scan rate of approximately 1° C. per minute under constant pressure.

Calorimetric data were analyzed using instrument-associated data analysis software to determine thermal unfolding profiles and associated transition temperatures. Control experiments were conducted to confirm that the presence of DMSO at the concentrations used did not materially affect the thermal denaturation behavior of TRPV1.

In the presence of capsaicin and representative compounds 12, 17, 21, and 24, TRPV1 exhibited reproducible multi-transition thermograms characterized by two principal thermal events, with transition temperatures and overall profile shapes that were qualitatively similar across these ligands.

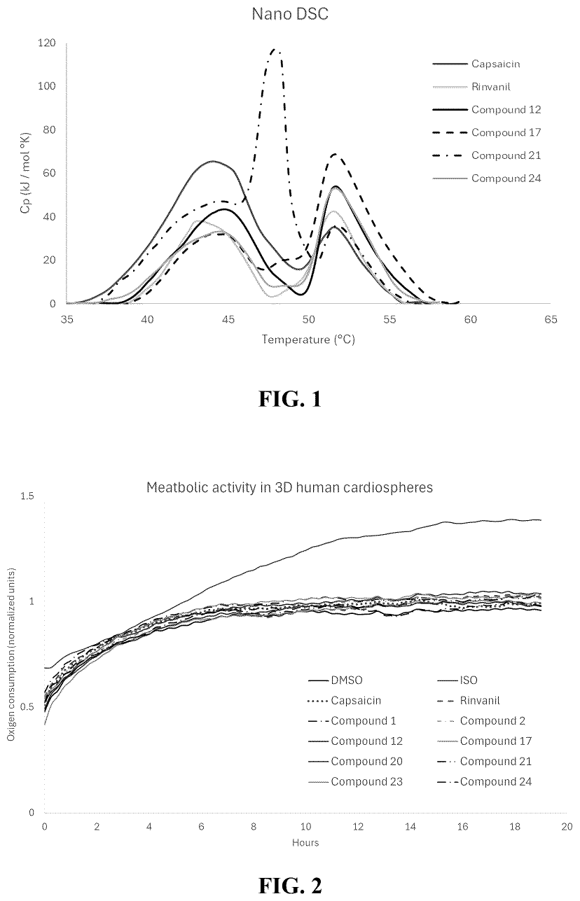

Several compounds produced thermograms that closely resembled the capsaicin control, indicating ligand-dependent modulation of TRPV1 thermal stability consistent with a shared interaction profile (FIG. 1).

In contrast, a structurally distinct comparator compound lacking a vanillylamine motif produced a thermogram with a markedly different peak morphology, including an altered relative contribution of the lower-temperature transition. This distinct thermodynamic signature is consistent with a different TRPV1 interaction profile relative to capsaicin and the compounds described herein.

Taken together with electrophysiological measurements, these thermodynamic data support that the compounds described herein engage TRPV1 in a reproducible and compound-dependent manner.

### Example B-7: Oxygen Consumption Rate (OCR) in 3D Human Adipocyte Organoids

Representative compounds were evaluated for metabolic activity using a three-dimensional human adipocyte organoid model. The organoids comprised differentiated human adipocytes organized in a 3D architecture that recapitulates key features of adipose tissue, including lipid storage and metabolic responsiveness.

Organoids were contacted with test compounds 30 days after the induction of cellular differentiation. A working concentration of 0.3 μM was established as standard (previously determined by a titration curve made with capsaicin as reference standard). Organoids were exposed for periods sufficient to elicit metabolic responses. Untreated organoids served as controls. All conditions were made by triplicates (n=3).

Table 2 shows the ΔOCR values (0-24 h) and AUC (0-48 h) expressed as percentage of the response to capsaicin 0.3 M (set as 100%). Data were normalized to the mean perimeter of the organoids and to the values of the control group (DMSO). Several compounds produced a measurable increase in the slope of oxygen consumption at 24 hours compared to untreated controls, indicating enhanced metabolic flux. In addition, treated organoids exhibited an increased oxygen consumption AUC over 48 hours, consistent with sustained activation of oxidative metabolism

TABLE 2
ΔOCR values (0-24 h) and AUC (0-48 h).

| Compound ID | AUC | ±STDEV | ±SEM | ΔOCR | ±STDEV | ±SEM |
|---|---|---|---|---|---|---|
| Capsaicin | 100.00 | 38.82 | 22.41 | 100.00 | 68.87 | 39.76 |
| Rinvanil | 75.71 | 13.40 | 7.74 | 84.98 | 12.25 | 7.08 |
| Compound 1 | 134.76 | 41.47 | 23.94 | 158.23 | 84.66 | 48.88 |
| Compound 2 | 135.46 | 35.36 | 20.41 | 209.07 | 82.56 | 47.67 |
| Compound 12 | 115.71 | 41.41 | 23.91 | 133.32 | 93.92 | 54.22 |
| Compound 17 | 120.99 | 40.02 | 23.11 | 109.77 | 70.16 | 40.50 |
| Compound 20 | 94.57 | 7.17 | 4.14 | 84.25 | 14.95 | 8.63 |
| Compound 21 | 119.32 | 53.19 | 30.71 | 149.23 | 103.84 | 59.95 |
| Compound 23 | 129.47 | 34.07 | 19.67 | 151.50 | 79.40 | 45.84 |
| Compound 24 | 93.53 | 4.95 | 2.86 | 90.14 | 9.02 | 5.21 |

### Example B-8: Organoid Morphology and Lipid Degradation

Structural and morphological changes in the 3D adipocyte organoids were monitored as an indirect measure of lipid mobilization. Following compound treatment, a reduction in organoid perimeter was observed relative to controls (DMSO), consistent with a decrease in intracellular lipid content and organoid compaction (Table 3).

TABLE 3
Perimeter reduction screen results expressed as percentage
of the DMSO group (set as 100%) after 48 h.

| Compound ID | (A/B) − (A/B DMSO)* | ΔP (z-score)** |
|---|---|---|
| Capsaicin | 1.5% | 1.94 |
| Rinvanil | 0.0% | −1.94 |
| Compound 1 | −0.2% | −0.34 |
| Compound 2 | −2.2% | −2.93 |
| Compound 12 | −0.6% | −0.77 |
| Compound 17 | −0.1% | −0.22 |
| Compound 20 | −1.8% | −2.52 |
| Compound 21 | −2.7% | −3.64 |
| Compound 23 | 0.0% | −0.34 |
| Compound 24 | −0.8% | −1.09 |

*Fold change of the sample (A/B) after and before 48 h of treatment minus same fold of control (DMSO). Data were normalized to the mean perimeter of the organoids.
**z-score of organoids perimeter reduction

### Example B-9: Glycerol Release

Lipolytic activity was further evaluated by measuring glycerol released into the culture medium following compound exposure. Treated organoids showed increased glycerol release compared to untreated controls, indicating enhanced triglyceride breakdown.

TABLE 4
Fold of glycerol release between the
compound tested and control (DMSO).

| Compound ID | Glycerol fold |
|---|---|
| Capsaicin | 1.09 |
| Rinvanil | 0.66 |
| Compound 1 | 1.64 |
| Compound 2 | 1.12 |
| Compound 12 | 0.68 |
| Compound 17 | 1.27 |
| Compound 20 | 1.13 |
| Compound 21 | 0.64 |
| Compound 23 | 0.86 |
| Compound 24 | 1.57 |

The combined increases in oxygen consumption, sustained metabolic activity over time, reduction in organoid size, and elevated glycerol release demonstrate that the disclosed compounds promote lipid mobilization and metabolic activation in human adipose tissue models.

### Example B-10: Cardiac Safety in Human 3D Cardiac Organoids

The cardiac safety profile of representative compounds of the invention was evaluated using three-dimensional human cardiac organoids comprising differentiated cardiac cells organized into spheroidal structures that recapitulate key aspects of human cardiac tissue function. The control human-induced Pluripotent Cell (hiPSC) line (IX0001, WCB1) from a normal donor was thawed and cultivated. A dual control was employed: a vehicle (baseline) control, and a positive physiological control using isoproterenol (ISO) at 0.1 μM. This ISO dose was selected because it produces a moderate and physiologically relevant increase in OCR (on the order of ~30-40% over baseline in our cardiospheres) without eliciting the signs of overt toxicity. This response defines an upper bound of safe metabolic activation in the assay. A working concentration of 0.3 M was established as the standard (previously determined by a titration curve made with capsaicin as reference standard). All conditions were made by triplicates (n=3).

### Metabolic Activity in Cardiac Organoids

Cardiac organoids were contacted with test compounds and compared to vehicle-treated controls (DMSO) and a positive physiological control using isoproterenol (ISO) at 0.1 μM. Oxygen consumption was monitored as a measure of cardiac metabolic activity at 24 hours (FIG. 2) and 48 hours (AUC) (Table 5) following compound exposure.

Table 5 shows the oxygen consumption rate AUC 0-48h (% ISO), normalized to DMSO (0%) and isoproterenol (ISO) 0.1 M (100%). ±SEM (n=3). Compounds are <20% ISO (SAFE). None reached ≥30%. No statistically significant deviations from DMSO. Sustained mitochondrial respiration is preserved. V-slope 12 h vs ISO of OCR (0-12 h), normalized to % ISO. Statistical testing vs DMSO and vs ISO. ISO: isoproterenol 0.1 μM. Test compounds cluster near DMSO slope, none reproduce ISO pattern. Acute OCR trajectory stable.

Treatment with compounds 12, 17-20, did not produce an increase in oxygen consumption relative to vehicle control at either time point, indicating preservation of basal cardiac metabolic function.

TABLE 5
AUC (0-48 h), ΔOCR values (0-24 h) and V-Slope of
tested compounds in human 3D cardiac organoids, normalized
to DMSO (0%) and isoproterenol (ISO) 0.1 μM (100%).

| Compound ID | Dose μM | AUC | ±SEM | ΔOCR | ±SEM | V-Slope | ±SEM |
|---|---|---|---|---|---|---|---|
| Isoproterenol | 0.1 | 100 | 0.83 | 100 | 0.05 | 100 | 0 |
| Capsaicin | 0.3 | 7.48 | 1.79 | 10.61 | 0.09 | −0.49 | 0 |
| Rinvanil | 0.3 | 10.41 | 0.51 | −37.77 | 0.01 | −22.21 | 0 |
| Compound 1 | 0.3 | 15.36 | 1.61 | −26.57 | 0.04 | −1.84 | 0 |
| Compound 2 | 0.3 | 11.65 | 0.35 | −23.79 | 0.04 | 2.84 | 0 |
| Compound 12 | 0.3 | 16.64 | 0.70 | −4.89 | 0.01 | 11.86 | 0 |
| Compound 17 | 0.3 | 5.09 | 0.22 | 51.79 | 0.04 | 17.98 | 0 |
| Compound 20 | 0.3 | 6.20 | 0.11 | 1.41 | 0.02 | 11.45 | 0 |
| Compound 21 | 0.3 | 6.20 | 0.11 | 1.41 | 0.02 | 11.45 | 0 |
| Compound 23 | 0.3 | 18.50 | 0.75 | −9.22 | 0.01 | 16.87 | 0 |
| Compound 24 | 0.3 | 8.89 | 1.02 | 13.15 | 0.08 | 0.41 | 0.01 |

Treatment with compounds did not produce an increase in oxygen consumption relative to vehicle control at either time point, indicating preservation of basal cardiac metabolic function.

### Visual Function Assessment

All wells were visually inspected using brightfield microscopy to evaluate spontaneous contractile activity in the cardiospheres after 48 hours of treatment. No cessation of beating was observed in any well, across all compounds. The maintenance of rhythmic contractions in all samples supports the conclusion that none of the treatments induced overt functional cardiotoxicity at the tested concentrations.

### 3D Human Organoids Cell Viability

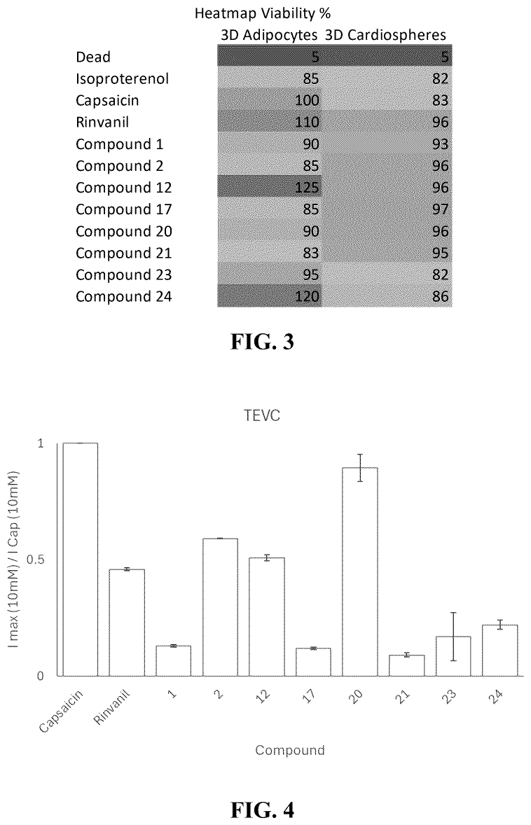

Cellular viability of the 3D human adipocytes and cardiospheres organoids was assessed following 48 hours of compound exposure measured by the CellTiter-Glo 3D assay. The results confirm that DMSO maintained viability around 100%, validating its role as the negative control, while the dead control produced the expected drastic reduction in viability, demonstrating assay sensitivity. Results can be seen in FIG. 3. Compounds 12 and 17-20 at 0.3 mM, isoproterenol at 1 M and capsaicin 0.3 μM, remained above the 80% cutoff, indicating that no overt cytotoxicity was observed under the tested conditions in human tissue models.

### Safety Interpretation

The absence of adverse effects on oxygen consumption and cellular viability in human 3D cardiac organoids suggests a favorable cardiac safety profile for the disclosed compounds. These results support the suitability of the compounds for systemic administration and therapeutic use.

### Example B-11: Electrophysiology in Xenopus laevis Oocytes

Functional activity of test compounds at TRPV1 was evaluated using two-electrode voltage clamp (TEVC) electrophysiology in Xenopus laevis oocytes.

Oocytes from Xenopus laevis were obtained from a commercial supplier and enzymatically defolliculated using collagenase (approximately 1 mg/mL) in ND96 buffer comprising 96 mM NaCl, 2 mM KCl, 5 mM HEPES, and 1 mM MgCl₂, adjusted to pH 7.4. Following defolliculation, oocytes were injected with messenger RNA encoding wild type.

TRPV1 was added using a nanoliter injector, with an injection amount of approximately 5 ng mRNA per oocyte.

Injected oocytes were incubated at approximately 16° C. in ND96 buffer supplemented with 2 mM CaCl₂) for a period of approximately 24-36 hours prior to electrophysiological recording.

Two-electrode voltage clamp recordings were performed using borosilicate glass microelectrodes filled with 3 M KCl. Whole-cell currents were recorded in an external recording solution containing 120 mM NaCl, 2 mM KCl, 2 mM MgCl₂, 1 mM EGTA, and 10 mM HEPES, adjusted to pH 7.4. Data acquisition was performed using a voltage-clamp amplifier, and recorded currents were analyzed using commercially available electrophysiology software.

### Electrophysiological Activity at TRPV1

Compounds were evaluated for functional activity at TRPV1 using two-electrode voltage clamp recordings in Xenopus laevis oocytes expressing wild-type TRPV1. Application of test compounds induced inward currents in TRPV1-expressing oocytes, whereas no appreciable currents were observed in non-injected control oocytes under identical conditions, indicating TRPV1-dependent activity.

Comparative experiments demonstrated that compounds 12, 17 and 21 elicited current responses with reduced maximal amplitudes relative to capsaicin when tested under comparable conditions, while maintaining channel activation (FIG. 4). The observed current profiles were consistent with partial agonist behavior at TRPV1. In some embodiments, TRPV1 activation by compounds of Formula (I), or a pharmaceutically acceptable salt thereof, occur at concentrations that produce measurable channel activation without inducing maximal channel opening.

These electrophysiological profiles demonstrate that the compounds are capable of modulating TRPV1 activity and support their use in regulating TRPV1-mediated physiological processes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound having the structure of Formula (I), or a pharmaceutically acceptable salt or solvate thereof: 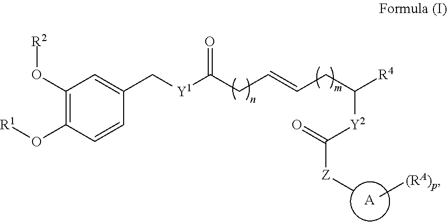
wherein;
Y¹ and Y² are each independently —O— or —NR³;
Z is absent, optionally substituted C₁-C₂ alkylene, —C═C—, —O—, or —NH—, wherein the alkylene is optionally substituted with —NH₂;
Ring A is an optionally substituted C₆-C₁₀ aryl, optionally substituted 5 to 10-membered heteroaryl, optionally substituted C₃-C₁₀ cycloalkyl, or optionally substituted C₃-C₁₀ heterocycloalkyl, each of which when substituted is substituted with 1 to 6 substituents selected from R^{A};
each R^{A} is independently selected from —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d};
R¹ is hydrogen or C₁-C₆ alkyl which is optionally substituted with 1 to 4 substituents selected from R^{e};
R² is hydrogen, optionally substituted C₁-C₆ alkyl, optionally substituted C₁-C₆ haloalkyl, optionally substituted C₁-C₆ heteroalkyl, or optionally substituted C₁-C₆ hydroxyalkyl, wherein the alkyl, haloalkyl, heteroalkyl, and hydroxyalkyl is optionally substituted with 1 to 4 substituents selected from R^{e};
each R^{e} is independently halogen, —NR^{a}R^{b}, —OR^{c}, or —SR^{a};
or R¹ and R² together with the atoms to which they are attached form a C₃-C₅ heterocycloalkyl;
each R³ is independently hydrogen or —CH₃;
R⁴ is C₄-C₂₀ alkyl;
each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ haloalkyl, optionally substituted C₃-C₁₀ cycloalkyl, and optionally substituted C₃-C₁₀ heterocycloalkyl, each of which is substituted with one or more R^{d};
or R^{a} and R^{b} together with the atoms to which they are attached form a C₃-C₁₀ heterocycloalkyl which is optionally substituted with one or more R^{d};
each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-C₁₀ heterocycloalkyl, optionally substituted C₆-C₁₀ aryl, and optionally substituted 5 to 10-membered heteroaryl, each of which is substituted with one or more R^{d};
or two R^{c} together with the atoms to which they are attached form a C₃-C₁₀ heterocycloalkyl which is optionally substituted with one or more R^{d};
each R^{d} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃;
m is 1, 2, or 3;
n is an integer from 5-8; and
p is 1-6.

2. (canceled)

3. The compound of claim 1, or a pharmaceutically acceptable salt or solvate thereof, wherein m is 1; and n is 6, 7, or 8.

4.-5. (canceled)

6. The compound of claim 1, or a pharmaceutically acceptable salt or solvate thereof, wherein Y¹ is —O—.

7. The compound of claim 1, or a pharmaceutically acceptable salt or solvate thereof, wherein Y¹ is —NR³—.

8.-9. (canceled)

10. The compound of claim 1, or a pharmaceutically acceptable salt or solvate thereof, wherein Ring A is an optionally substituted C₃-C₁₀ cycloalkyl or optionally substituted C₃-C₁₀ heterocycloalkyl, each of which when substituted is substituted with 1 to 6 substituents selected from R^{A}.

11. The compound of claim 1, or a pharmaceutically acceptable salt or solvate thereof, wherein Ring A is an optionally substituted 5 to 10-membered heteroaryl comprising 1, 2, or 3 heteroatoms selected from 0 and N, which is optionally substituted with 1 to 6 substituents selected from R^{A}.

12.-13. (canceled)

14. The compound of claim 1, or a pharmaceutically acceptable salt or solvate thereof, wherein Ring A is an optionally substituted C₆-C₁₀ aryl, which is substituted with 1 to 6 substituents selected from R^{A}, and wherein when Ring A is phenyl then the phenyl is substituted.

15.-24. (canceled)

25. The compound of claim 1, wherein the compound is of Formula (III), or a pharmaceutically acceptable salt or solvate thereof: 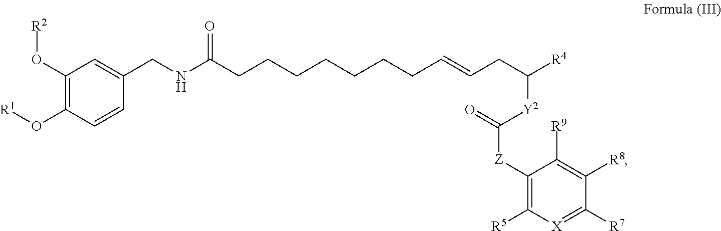
wherein;
X is N or CR⁶;
Y² is —O— or —NR³;
Z is absent, optionally substituted C₁-C₂ alkylene, —C═C—, —O—, or —NH—, wherein the alkylene is optionally substituted with —NH₂;
R¹ is hydrogen or C₁-C₆ alkyl which is optionally substituted with 1 to 4 substituents selected from R^{e};
R² is hydrogen, optionally substituted C₁-C₆ alkyl, optionally substituted C₁-C₆ haloalkyl, optionally substituted C₁-C₆ heteroalkyl, or optionally substituted C₁-C₆ hydroxyalkyl, wherein the alkyl, haloalkyl, heteroalkyl, and hydroxyalkyl is optionally substituted with 1 to 4 substituents selected from R^{e};
or R¹ and R² together with the atoms to which they are attached form a C₃-C₅ heterocycloalkyl;
each R^{e} is independently halogen, —NR^{a}R^{b}, —OR^{c}, or —SR^{a};
R³ is hydrogen or —CH₃;
R⁴ is C₄-C₂₀ alkyl;
R⁵, R⁶, R⁷, R⁸, and R⁹ are each independently hydrogen, —CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, C₃-C₁₀ cycloalkyl, C₃-C₁₀ heterocycloalkyl, C₆-C₁₀ aryl, or 5 to 10-membered heteroaryl, wherein each alkyl, haloalkyl, heteroalkyl, cycloalkyl, heterocycloalkyl, aryl, and heteroaryl is optionally substituted with 1 to 4 substituents selected from R^{d}; wherein at least one of R⁵, R⁶, R⁷, R⁸, or R⁹ is not hydrogen;
or R⁵ and R⁶ or R⁶ and R⁷ or R⁷ and R⁸ together with the atoms to which they are attached form an optionally substituted C₃-C₆ cycloalkyl, optionally substituted C₃-C₆ heterocycloalkyl, optionally substituted C₆-C₈ aryl, or optionally substituted 5 to 6-membered heteroaryl, each of which is optionally substituted with 1 to 4 substituents selected from R^{d};
each R^{a} and R^{b} is independently selected from hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ haloalkyl, optionally substituted C₃-C₁₀ cycloalkyl, and optionally substituted C₃-C₁₀ heterocycloalkyl, each of which is substituted with one or more R^{d};
or R^{a} and R^{b} together with the atoms to which they are attached form a C₃-C₁₀ heterocycloalkyl which is optionally substituted with one or more R^{d};
each R^{c} is independently selected from hydrogen, optionally substituted C₁-C₆ alkyl, optionally substituted C₂-C₆ alkenyl, optionally substituted C₁-C₆ heteroalkyl, optionally substituted C₁-C₆ haloalkyl, optionally substituted C₃-C₁₀ cycloalkyl, optionally substituted C₃-C₁₀ heterocycloalkyl, optionally substituted C₆-C₁₀ aryl, and optionally substituted 5 to 10-membered heteroaryl, each of which is substituted with one or more R^{d};
or two R^{c} together with the atoms to which they are attached form a C₃-C₁₀ heterocycloalkyl which is optionally substituted with one or more R^{d}; and
each R^{d} is independently selected from halogen, —OH, —CH₃, —CF₃, —OCH₃, —NH₂, —NHCH₃, —N(CH₃)₂, —C(O)OH, —C(O)OCH₃, —C(O)NH₂, —C(O)NHCH₃, —C(O)N(CH₃)₂, —NHC(O)OH, —OC(O)NH₂, and —NHC(O)CH₃.

26. The compound of claim 25, or a pharmaceutically acceptable salt or solvate thereof, wherein X is CR⁶.

27. The compound of claim 25, or a pharmaceutically acceptable salt or solvate thereof, wherein R⁵, R⁶, R⁷, R⁸, and R⁹ are each independently hydrogen, CN, —NO₂, —NR^{a}R^{b}, —OR^{c}, —SR^{a}, —C(O)R^{c}, —C(O)OR^{c}, —C(O)NR^{a}R^{b}, C₁-C₆ alkyl, C₁-C₆ haloalkyl, C₁-C₆ heteroalkyl, or C₁-C₆ hydroxyalkyl, wherein each alkyl, haloalkyl, heteroalkyl, and hydroxyalkyl is optionally substituted with 1 to 4 substituents selected from R^{d}.

28. (canceled)

29. The compound of claim 27, or a pharmaceutically acceptable salt or solvate thereof, wherein R⁵ and R⁹ are each hydrogen; and R⁶, R⁷, and R^{c} are each independently hydrogen or —OR^{c}, wherein each R^{c} is independently selected from hydrogen, optionally substituted C₁-C₆ alkyl, or optionally substituted C₁-C₆ haloalkyl.

30.-43. (canceled)

44. The compound of claim 1, or a pharmaceutically acceptable salt or solvate thereof, wherein Z is absent or —C═C—.

45.-46. (canceled)

47. The compound of claim 1, or a pharmaceutically acceptable salt or solvate thereof, wherein Y² is —O—.

48.-55. (canceled)

56. The compound of claim 1, or a pharmaceutically acceptable salt or solvate thereof, wherein R¹ is hydrogen and R² is —CH₃.

57. (canceled)

58. The compound of claim 1, or a pharmaceutically acceptable salt or solvate thereof, wherein R¹ and R² together with the atoms to which they are attached form a 1,3-dioxolane.

59. (canceled)

60. The compound of claim 1, or a pharmaceutically acceptable salt or solvate thereof, wherein R⁴ is C₄-C₁₀ alkyl.

61.-65. (canceled)

66. The compound of claim 1, wherein the compound is: 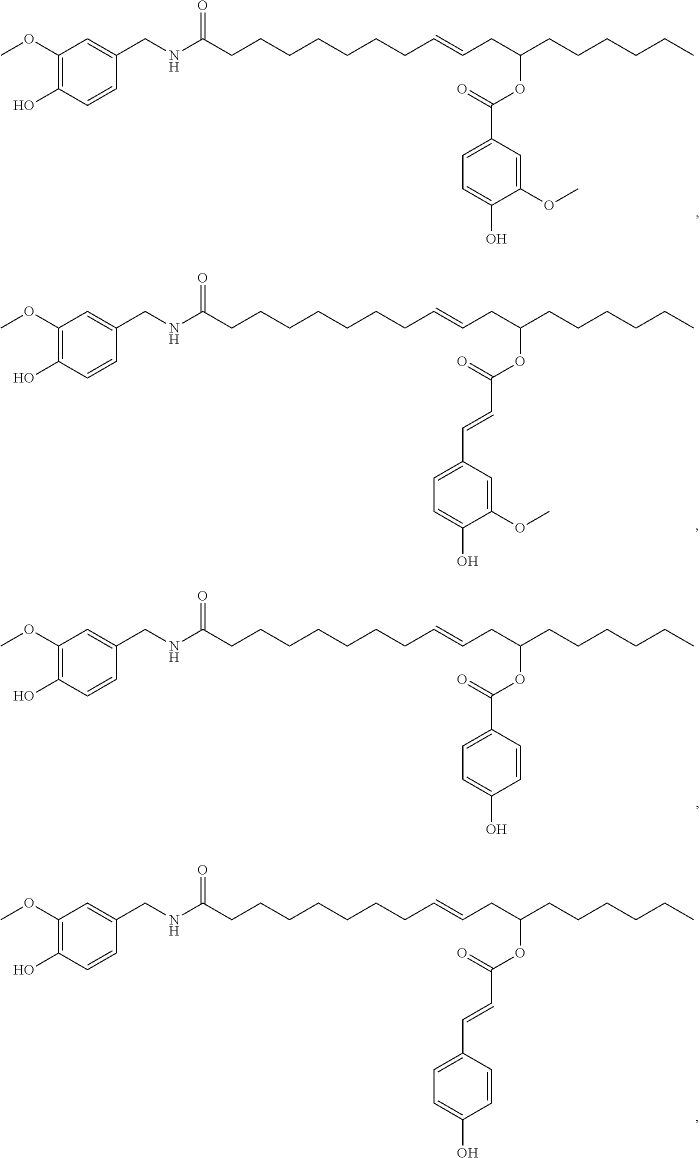 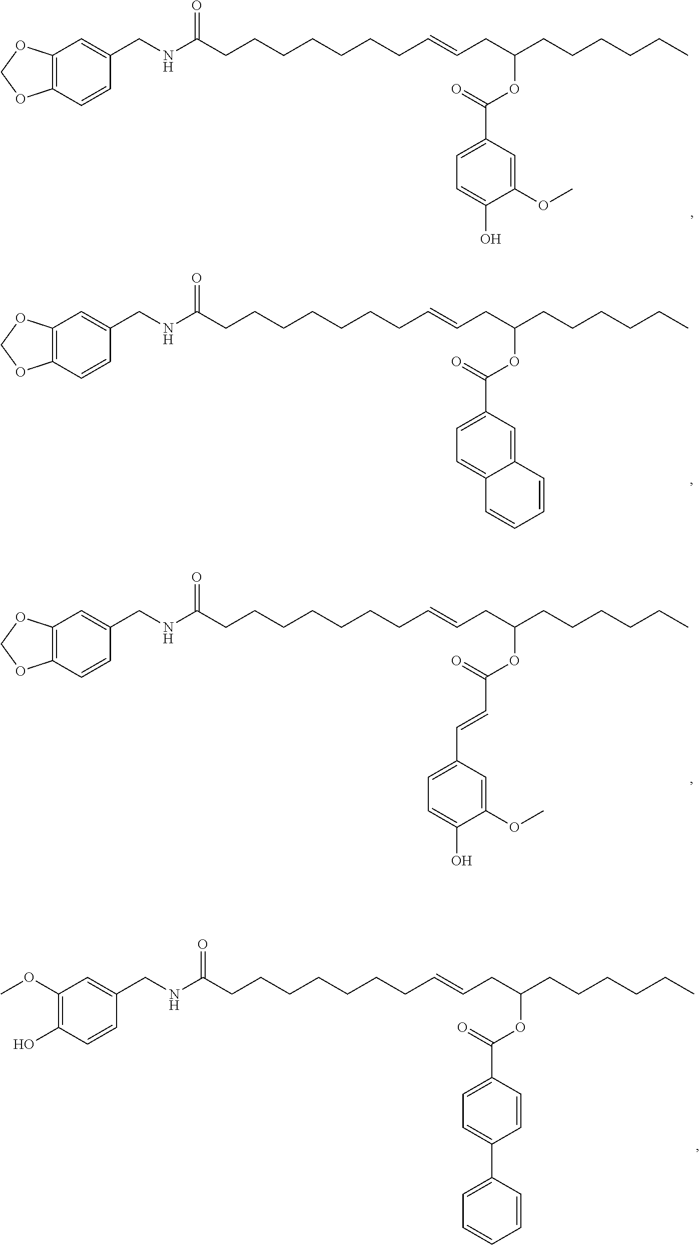 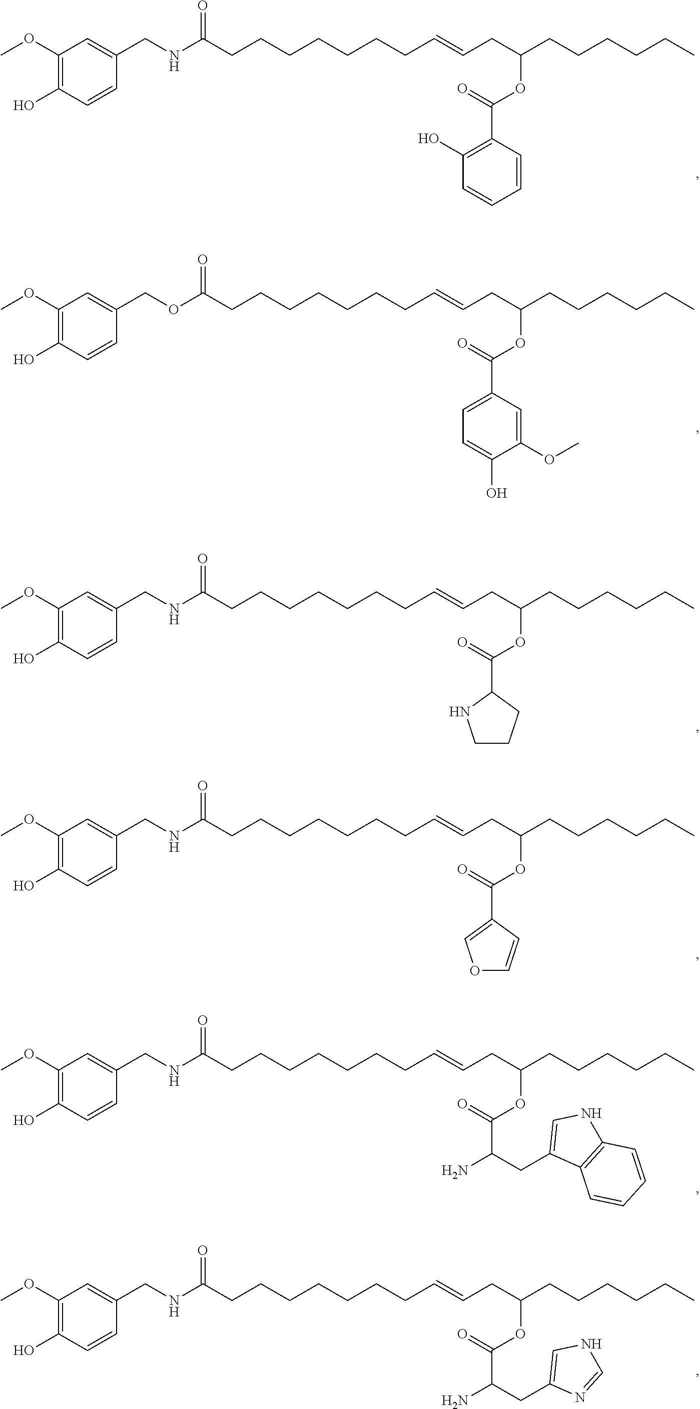 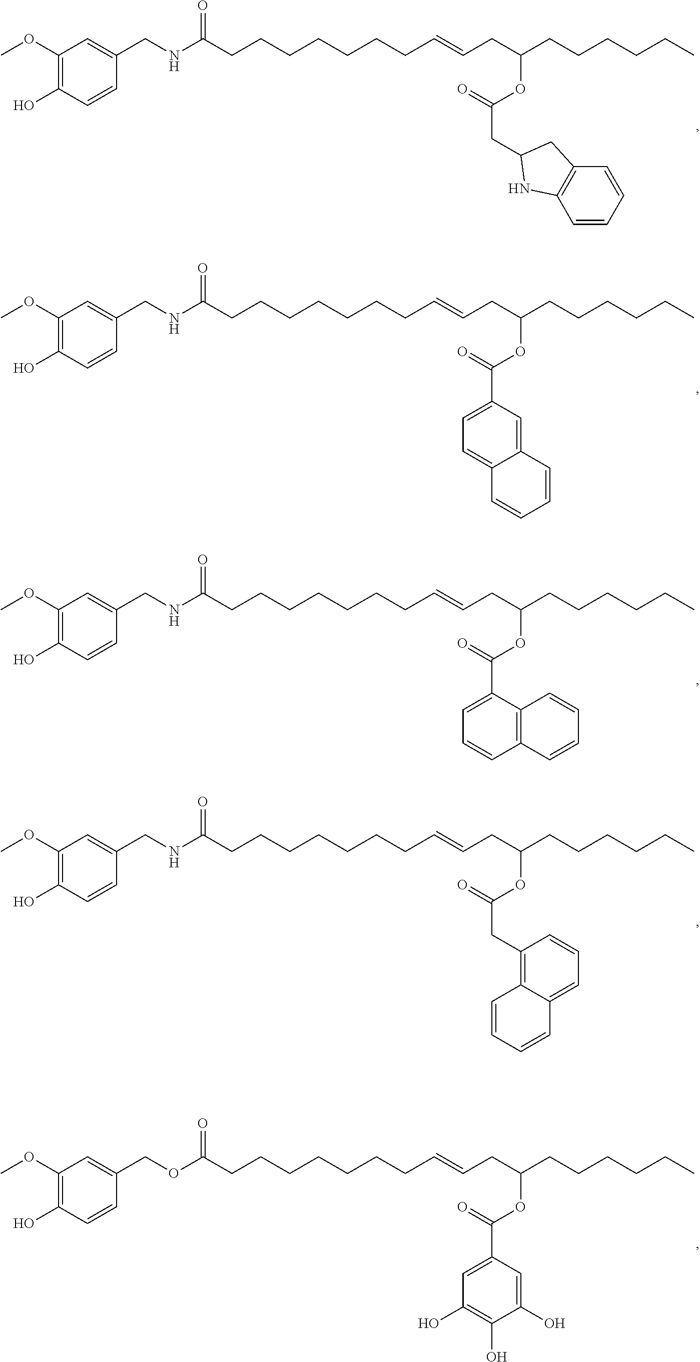 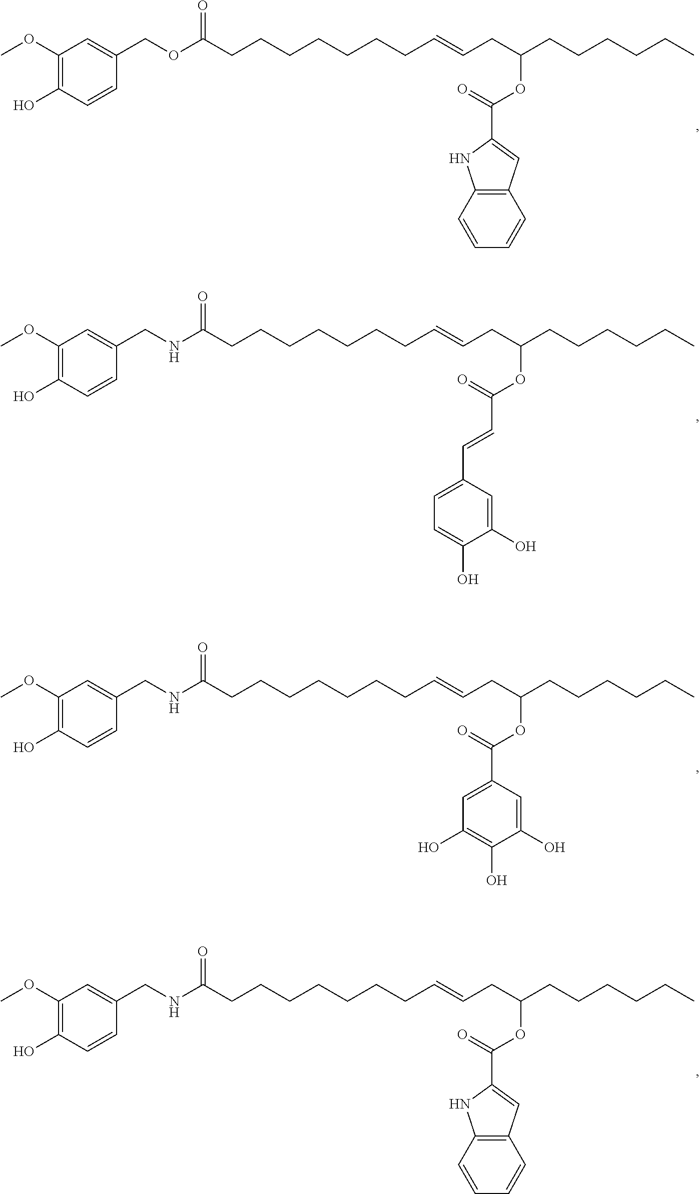
or a pharmaceutically acceptable salt or solvate thereof.

67. A pharmaceutical composition comprising the compound of claim 1, or a pharmaceutically acceptable salt or solvate thereof, and a pharmaceutically acceptable excipient.

68. A method of treating a disease or condition associated with agonism of transient receptor potential vanilloid 1 (TRPV1) in a subject in need thereof, comprising administering to the subject a compound of claim 1, or a pharmaceutically acceptable salt or solvate thereof.

69. A method of treating a metabolic condition in a subject in need thereof, the method comprising administering to the subject a therapeutically effective amount of a compound of claim 1, or a pharmaceutically acceptable salt or solvate thereof.

70.-71. (canceled)
